(19) United States
(12) Patent Application Publication
(10) Pub. No.: US 2026/0102068 A1
(43) Pub. Date: Apr. 16, 2026

(54) DETECTING BLOOD PRESSURE DURING CHEST COMPRESSIONS

(71) Applicant: Stryker Corporation, Portage, MI (US)

(72) Inventors: Tyson G. Taylor, Bothell, WA (US); Rose Tingwei Yin, Bellevue, WA (US); Fred W. Chapman, Newcastle, WA (US); Robert G. Walker, Seattle, WA (US)

(73) Assignee: Stryker Corporation, Portage, MI (US)

(21) Appl. No.: 19/358,001

(22) Filed: Oct. 14, 2025

Related U.S. Application Data
(60) Provisional application No. 63/706,931, filed on Oct. 14, 2024.

(51) Int. Cl. A61B 5/021; A61B 5/00; A61B 5/0215; A61H 31/00

(52) U.S. Cl. CPC A61B 5/02108; A61B 5/0215; A61B 5/4836; A61B 5/742; A61H 31/005

(57) ABSTRACT

An example method includes detecting a blood pressure waveform of a blood vessel of a subject receiving chest compressions; identifying an interpulse interval of the blood pressure waveform, s; and determining a diastolic pressure of the subject by determining a mean of the interpulse interval of the blood pressure waveform or a mean of a portion of the interpulse interval of the blood pressure waveform. The interpulse interval extends between pulse waves transmitted through the blood vessel and caused by the chest compressions.

### CROSS-REFERENCE TO RELATED APPLICATION

This application claims priority to U.S. Provisional App. No. 63/706,931, which was filed on Oct. 14, 2024 and is incorporated by reference herein in its entirety.

### BACKGROUND

When an subject experiences cardiac arrest, their heart is unable to effectively pump blood throughout their body. Some instances of cardiac arrest are caused by cardiac arrhythmias, such as ventricular fibrillation (VF). VF is treatable by administering a high-energy electrical shock to the heart, such as by a defibrillator. However, it may take some time for a rescuer or defibrillator to identify that the subject has VF, and not some other cause of cardiac arrest.

While the subject remains in cardiac arrest, the subject's brain and other vital organs are unable to receive sufficient oxygenation due to the subject's lack of spontaneous blood circulation. This may cause the subject to lose consciousness. If the subject remains in cardiac arrest for a sufficient amount of time without treatment, the subject may experience a serious hypoxic injury, such as brain injury or death.

Cardiopulmonary resuscitation (CPR) can delay or prevent the subject from developing a hypoxic injury. CPR includes the administration of chest compressions to the subject, which can produce some blood flow throughout the subject's circulatory system even when the heart of the subject is unable to spontaneously pump blood.

### BRIEF DESCRIPTION OF THE DRAWINGS

FIG. 1 illustrates an example environment for detecting a clinically relevant blood pressure measurement or indication of spontaneous circulation during chest compression administration.

FIG. 2 illustrates a comparison between a blood pressure waveform and a chest compression waveform.

FIG. 3 illustrates an example process for determining a diastolic blood pressure of a subject receiving chest compressions.

FIG. 4 illustrates an example process for identifying spontaneous circulation in a subject receiving chest compressions.

FIG. 5 illustrates an example of a blood pressure sensor configured to detect an instantaneous blood pressure of a subject.

FIG. 6 illustrates an example of an external defibrillator configured to perform various functions described herein.

FIG. 7 illustrates a chest compression device configured to perform various functions described herein.

### DETAILED DESCRIPTION

Chest compressions are an important medical intervention in emergency medical situations. For example, a rescuer responding to the scene of an subject in cardiac arrest will prioritize administering chest compressions before taking steps to identify and treat the cause of the cardiac arrest. However, chest compressions can complicate patient monitoring.

In particular, it can be difficult to identify a clinically useful measurement of the subject's blood pressure, or other indicator of spontaneous blood circulation, when the subject is receiving chest compressions. Conventional algorithms for identifying diastolic blood pressure when a heart is spontaneously beating, for example, involve identifying a minimum blood pressure during a diastolic phase of the cardiac cycle. When chest compressions are applied to a subject (with or without a spontaneously beating heart), the “decompression” phase of the chest compressions is similar to the diastolic phase of the cardiac cycle. However, the minimum blood pressure corresponding to the decompression phase of the chest compressions does not correspond to a clinically meaningful blood pressure measurement. For example, the minimum blood pressure corresponding to the decompression phase can be caused by chest compression artifact, rather than a physiologically accurate measurement of blood pressure. While it may be possible to obtain a clinically relevant measurement of blood pressure or blood circulation when the chest compression artifact is absent during a chest compression pause, such pauses can cause harm to the subject. For instance, if the subject lacks spontaneous circulation, a pause in chest compressions can increase the risk of developing a hypoxic injury, or increase the severity of a hypoxic injury. Accordingly, there is a need to apply a different methodology for determining diastolic blood pressure, or its equivalents, when chest compressions are applied to the subject.

Implementations of the present disclosure address these and other problems by accurately identifying a clinically relevant estimate of the diastolic blood pressure of the subject when the subject is receiving chest compressions. In various cases, a monitor identifies an interpulse interval of a blood pressure waveform of a blood vessel of the subject. The interpulse interval, for instance, corresponds to a decompression interval of chest compressions administered to the subject. The monitor estimates the diastolic blood pressure by analyzing the interpulse interval. For example, the monitor calculates the diastolic blood pressure by determining a mean of a third, or two-thirds, of the interpulse interval.

Implementations of the present disclosure can also be used to accurately identify a return of spontaneous circulation (ROSC) when the subject is receiving chest compressions. In various cases, the monitor is configured to analyze multiple portions of the interpulse interval of the blood pressure waveform. For example, the monitor determines means of respective portions of the interpulse interval. If a difference between the means is sufficiently high, the monitor infers that the subject has spontaneous circulation. Accordingly, the monitor may efficiently identify when the chest compressions are no longer necessary.

Various implementations of the present disclosure are directed to improvements in the field of medical diagnostics and treatments. By determining the diastolic blood pressure during an interpulse interval, implementations of the present disclosure provide more accurate and reliable blood pressure measurements. Moreover, some implementations described herein enable ROSC identification without requiring a pause in chest compressions. Because no pause in chest compressions is necessary to identify ROSC, various implementations of the present disclosure can efficiently identify the condition of a subject while minimizing the risk of hypoxic injury.

Various implementations of the present disclosure will now be described with reference to the accompanying figures.

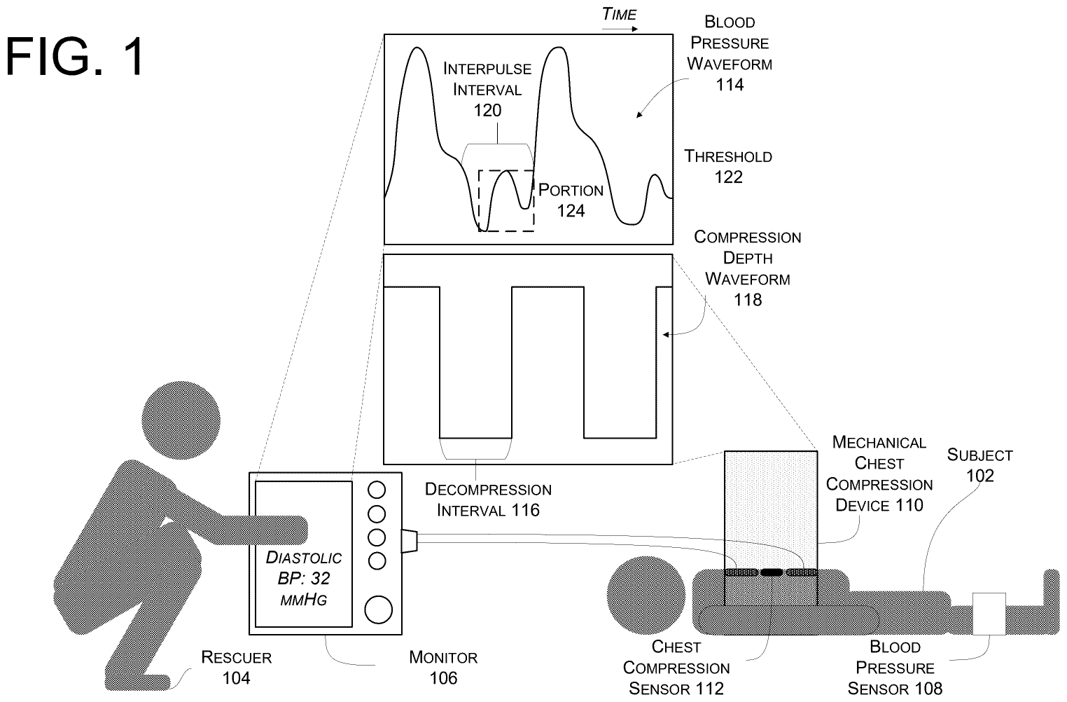

FIG. 1 illustrates an example environment 100 for detecting a clinically relevant blood pressure measurement or indication of spontaneous circulation during chest compression administration. In the environment 100, a subject 102 is experiencing a medical emergency. For instance, the subject 102 may have unexpectedly collapsed. The environment 100, in some cases, is a non-clinical environment, such as a school, an office, or an airport terminal. In some examples, the environment 100 is a clinical environment, such as a hospital. In various cases, the subject 102 has lost consciousness due to cardiac arrest. For example, the subject 102 has developed a cardiac arrhythmia that prevents the heart of the subject 102 from effectively pumping blood throughout the body of the subject 102. As a result, the brain of the subject 102 has not received sufficient oxygenation to sustain consciousness of the subject 102.

A rescuer 104 has been deployed to the environment 100 to monitor the condition of the subject 102 and, if necessary, administer an emergency treatment to the subject 102. In some cases, the rescuer 104 is a bystander. In some examples, the rescuer 104 has specialized medical training. For instance, the rescuer 104 has been deployed to the environment 100 in response to the medical emergency of the subject 102. For instance, the rescuer 104 is an emergency medical technician (EMT), physician, nurse, or other clinical care provider.

In various cases, the rescuer 104 utilizes a monitor 106 to assess the condition of the subject 102. The monitor 106 is configured to detect one or more physiological parameters of the subject 102. The term “physiological parameter” refers to a metric detected from a body of a subject that is relevant to a condition of the subject. Examples of physiological parameters include electrocardiogram (ECG), electroencephalogram (EEG), heart rate, pulse rate, an airway parameter (e.g., a tidal volume, airway pressure, respiratory rate, partial pressure of CO₂ in an airway, partial pressure of O₂ in the airway, etc.), temperature, blood pressure, blood oxygenation (e.g., pulse oxygenation, photoplethysmography, etc.), blood flow parameters (e.g., blood velocity, volumetric flow rate, etc.) and the like. In some examples, the monitor 106 is communicatively coupled with one or more sensors configured to detect the physiological parameter(s). Examples of the sensors include electrodes (e.g., external electrodes disposed on the skin of the subject 102, such as ECG electrodes or EEG electrodes), heart rate sensors, pulse rate sensors, gas sensors, respiration sensors, thermometers, blood pressure sensors, blood oxygenation sensors, and the like. In some cases, the monitor 106 is further configured to administer a treatment to the subject 102. The treatment, for instance, includes one or more types of electrotherapies. In particular examples, the monitor 106 is a monitor-defibrillator configured to externally administer an electrical shock to the subject 102, pacing pulses, synchronized cardioversion, or any combination thereof. The monitor 106 is portable, in various cases.

The monitor 106, in various implementations, is communicatively coupled with a blood pressure sensor 108 that is configured to sample a blood pressure of the subject 102. The blood pressure sensor 108 is configured to exchange data with the monitor 106 over one or more communication interfaces. The communication interface(s) include one or more wired interfaces, one or more wireless interfaces (e.g., a BLUETOOTH™ interface), or a combination thereof. In various examples, the blood pressure sensor 108 is configured to sample a fluid pressure within at least one blood vessel of the subject 102. The blood pressure sensor 108 is configured to transmit communication signals indicative of the sampled pressure to the monitor 106 over the communication interface(s). The communication signals, in various cases, convey a stream of data representing the sampled pressure substantially in real time after detection by the blood pressure sensor 108.

In various cases, the subject 102 presents without spontaneous circulating blood flow. To reduce the risk of the subject 102 developing a hypoxic injury due to their lack of spontaneous circulation, the rescuer 104 causes chest compressions to be administered to the subject 102. In the example illustrated in FIG. 1, a mechanical chest compression device 110 is configured to administer the chest compressions to the subject 102. For example, the mechanical chest compression device 110 includes a motor that periodically moves a compressor (e.g., a plunger or band) configured to compress the chest of the subject 102. In some cases, the mechanical chest compression device 110 is configured to control and/or detect the state of the compressor, such that the mechanical chest compression device 110 is configured to identify various parameters of the chest compressions being administered to the subject 102. The term “chest compression parameter,” and its equivalents, refers to any metric characterizing chest compressions applied to a subject. Examples of chest compression parameters include, for instance, a depth of the chest compressions, a frequency of the chest compressions, a duty cycle of the chest compressions, a velocity of the chest compressions, and a position at which the chest compressions are administered to the chest of the subject 102.

In some implementations, a chest compression sensor 112 is disposed on the chest of the subject 102 and is configured to detect one or more parameters of the chest compressions administered to the subject 102 by the mechanical chest compression device 110. In some cases, the chest compression sensor 112 includes a pressure sensor configured to detect an amount and timing of pressure applied to the chest of the subject 102 by the mechanical chest compression device 110. In some examples, the chest compression sensor 112 includes multiple electrodes configured to detect a transthoracic impedance of the subject 102. Based on the transthoracic impedance, the chest compression sensor 112 is configured to detect the timing of the chest compressions. Although not specifically illustrated in FIG. 1, in some cases, the rescuer 104 administers manual chest compressions to the subject 102. The chest compression sensor 112, in various cases, detects the chest compressions manually administered to the subject 102. The monitor 106, the chest compression device 110, and the chest compressions sensor 112 are communicatively coupled to one another via one or more communication interfaces.

The chest compressions administered to the subject 102 can make it difficult to detect clinically relevant blood pressure parameters of the subject 102. Examples of blood pressure parameters include instantaneous blood pressure, diastolic blood pressure (or “diastolic pressure”), systolic blood pressure (or “systolic pressure”), pulse pressure, and mean arterial pressure (MAP). In examples in which the heart of the subject 102 is spontaneously circulating blood and the subject 102 is not receiving chest compressions, the diastolic pressure of the subject 102 is typically measured as the minimum blood pressure in one full cardiac cycle (e.g., one heartbeat) from systole to diastole. However, in the example illustrated in FIG. 1, the lack of spontaneous circulating blood in the body of the subject 102, as well as the administration of the chest compressions, result in an a blood pressure pattern detected by the blood pressure sensor 108 that is distinct from a blood pressure pattern detected by the blood pressure sensor 108 when the subject 102 has spontaneous circulation and is not receiving chest compressions.

FIG. 1 illustrates a blood pressure waveform 114 generated based on instantaneous blood pressure measurements taken over time by the blood pressure sensor 108. Changes in the blood pressure waveform 114 over time are the result, at least in part, of the chest compressions administered to the subject 102. In various cases, the mechanical chest compression device 110 is configured to periodically administer the chest compressions in a chest compression cycle. Each chest compression cycle includes a compression phase and a decompression phase. The compression phase is administered during a compression interval and the decompression phase is administered during a decompression interval 116. A ratio of the compression interval to the decompression interval 116 is referred to as the duty cycle of the chest compressions. A chest compression waveform 118 illustrated in FIG. 1 shows the depth of chest compressions administered to the subject 102 by the mechanical chest compression device 110. In the example illustrated in FIG. 1, the mechanical chest compression device 110 administers the chest compressions at a 1:1 duty cycle, however, implementations are not so limited. In FIG. 1, the blood pressure waveform 114 and the compression depth waveform 118 are vertically aligned with respect to time.

As illustrated in FIG. 1, the blood pressure waveform 114 includes maxima that correspond to the compression phases of the chest compressions. Physiologically, the compressions administered by the mechanical chest compression device 110 create pulse waves that travel through the blood vessels of the subject 102. When the pulse waves reach the blood vessel being monitored by the blood pressure sensor 108, peaks are generated in the blood pressure waveform 114. Between the pulse waves, the blood pressure of the subject 102 decreases due to the decompression phases of the mechanical chest compression device 110, and the blood pressure waveform 114 may exhibit one or more local minima. However, in various cases, the local minima between the pulse waves can be the result of artifact caused by the chest compressions, rather than an accurate estimation of the ground truth blood pressure of the subject 102.

For example, as illustrated in FIG. 1, the blood pressure waveform 114 exhibits deep valleys immediately after the compression peak. In some cases, these valleys correspond to an artifact in the blood pressure waveform 114 due to the chest recoiling (or being lifted) during the decompression phase. As another example, in some cases, the blood pressure waveform 114 exhibits a periodic, “ringing” artifact during the decompression phase.

Accordingly, it is difficult to detect the diastolic pressure of the subject 102 by merely identifying the minimum blood pressure between peaks of the blood pressure waveform 114. Moreover, the absolute minimum between peaks of the blood pressure waveform 114 is not necessarily reflective of a minimum, physiologically relevant blood pressure measurement caused by the decompression phase (also referred to herein as the diastolic pressure of the subject 102, regardless of whether the subject 102 has a spontaneous cardiac cycle with a diastolic phase).

Various implementations of the present disclosure address these and other problems by providing a novel technique for accurately detecting the diastolic pressure of the subject 102 receiving chest compressions based on the blood pressure waveform 114. In various cases, the monitor 106 is configured to detect the diastolic pressure based on the blood pressure waveform 114 detected during the administration of chest compressions to the subject 102. However, it can be noted that the techniques described herein can be performed by the blood pressure sensor 108 itself, or the mechanical chest compression device 110 if the blood pressure waveform 114 is provided to the mechanical chest compression device 110.

In some examples, the monitor 106 switches between a first mode, in which the monitor 106 reports the minimum blood pressure during the diastolic phase as the diastolic blood pressure, and a second mode, in which the monitor 106 reports a different estimate of the diastolic blood pressure (as described herein). The monitor 106, for instance, activates the second mode in response to detecting that chest compressions are administered to the subject 102. In various cases, the monitor 106 activates the first mode in response to detecting that chest compressions are not being administered to the subject 102.

In various cases, the monitor 106 is configured to detect an interpulse interval 120 of the blood pressure waveform 114. The interpulse interval 120 is the portion of the blood pressure waveform 114 that corresponds to the decompression interval 116 of the chest compressions. Because the pulse waves caused by the chest compressions must travel between the chest of the subject and the blood vessel being monitored by the blood pressure sensor 108, the interpulse interval 120 is delayed with respect to the decompression interval 116.

In some cases, the interpulse interval 120 is identified by determining a segment of the blood pressure waveform 114 that is below a threshold 122. The threshold 122, for instance, is a mean of at least a segment of the blood pressure waveform 114. For instance, the threshold 122 is determined by calculating an integral of the blood pressure waveform 114 over a time interval (e.g., an area underneath a segment of the blood pressure waveform 114 defined over the time interval) and dividing the integral by the length of the time interval. In various cases, a beginning or start of the interpulse interval 120 is defined as a transition in the blood pressure waveform 114 from above the threshold 122 to below the threshold 122. An ending of the interpulse interval 120 is defined as a transition in the blood pressure waveform 114 from below the threshold 122 to above the threshold 122.

Other techniques may be utilized to identify the interpulse interval 120. In some cases, the duration or length of the decompression interval 116 is presumed to be equivalent to the length or duration of the interpulse interval 120. The length of the decompression interval 116 is detected by the chest compression sensor 112, in some examples, In some cases, the decompression interval 116 is identified based on a communication signal received from the chest compression device 110. In some cases, the decompression interval 116 is predetermined by the monitor 106. For example, the decompression interval 116 is a fixed value. In various cases, the center of the interpulse interval 120 is identified. In some examples, the center of the interpulse interval 120, for instance, may be defined as an absolute minimum between adjacent peaks of the blood pressure waveform 114. In some implementations, the monitor 106 generates a moving average of the blood pressure waveform 114. For instance, the monitor 106 applies a boxcar filter to the blood pressure waveform 114 and/or generates a Gaussian moving average of the blood pressure waveform 114. A window of the moving average (e.g., a width of the boxcar filter) is equal to, or proportional to, the length of the decompression interval 116. In various cases, the moving average of the blood pressure waveform 114 includes a single minimum corresponding to each instance of the decompression interval 116. According to some implementations, the center of the interpulse interval 120 is determined by identifying a local minimum of the moving average of the blood pressure waveform 114.

In some implementations, the monitor 106 is configured to identify the beginning of the interpulse interval 120 by subtracting half of the length of the decompression interval 116 from the time at which the center of the interpulse interval 120 occurs, and is configured to identify the ending of the interpulse interval 120 by adding half of the length of the decompression interval 116 from the time at which the center of the interpulse interval 120 occurs.

The monitor 106 is configured to calculate the diastolic blood pressure by analyzing the interpulse interval 120 of the blood pressure waveform 114. In some cases, the monitor 106 is configured to determine the diastolic blood pressure by determining a mean of the interpulse interval 120. For instance, the monitor 106 is configured to divide an integral of the blood pressure waveform 114 across the interpulse interval 120 by a duration of the interpulse interval 120.

In some cases, the monitor 106 is configured to determine the diastolic pressure based on a portion 124 of the interpulse interval 120. The portion 124 is in a range of 0.01% to 99.99% of the interpulse interval 120, such as in a range of a third of the interpulse interval 120 to two-thirds of the interpulse interval 120. In some cases, the portion 124 begins simultaneously with the beginning of the interpulse interval 120. In some examples, the portion 124 ends simultaneously with an end of the interpulse interval 120. In various implementations, the diastolic pressure of the subject 102 is calculated as a mean of the portion 124 of the interpulse interval 120.

In various implementations, the monitor 106 outputs the diastolic pressure. For instance, the monitor 106 includes a display that visually presents an indication of the diastolic pressure. The rescuer 104, for instance, recognizes the condition of the subject 102 by ascertaining the diastolic pressure. In some examples, the monitor 106 outputs the diastolic pressure to the rescuer 104 audibly via one or more speakers. According to some cases, the monitor 106 also outputs the blood pressure waveform 114 to the rescuer 104, such as by visually presenting the blood pressure waveform 114 substantially in real time as the blood pressure sensor 108 is sampling the blood pressure of the blood vessel of the subject 102.

In some cases, the monitor 106 generates an alert based on the diastolic pressure. For instance, the monitor 106 generates the alert in response to determining that the diastolic pressure is greater than an upper threshold or under a lower threshold. In particular cases, the monitor 106 infers that the chest compressions applied to the subject 102 are inadequate by determining that the diastolic pressure is below the lower threshold. In various cases, the monitor 106 outputs the alert to the rescuer 104 visually via the display, audibly via the speaker(s), or via some other output device. For instance, the alert includes an instruction to change a parameter of the chest compressions (e.g., a chest compression depth, position on the chest, frequency, duty cycle, etc.) in order to increase the efficacy of the chest compressions.

In some examples, the monitor 106 communicates and/or controls the mechanical chest compression device 110 based on the diastolic blood pressure and/or the blood pressure waveform 114. For example, the monitor 106 outputs an indication of the diastolic blood pressure and/or the alert to the mechanical chest compression device 110. According to various implementations, the mechanical chest compression device 110 is configured to modify one or more chest compression parameters of the chest compressions based on the diastolic pressure and/or the alert. For instance, the mechanical chest compression device 110 changes a depth of the chest compressions, a frequency of the chest compressions, a duty cycle of the chest compressions, a velocity of the chest compressions, and a position at which the chest compressions are administered to the chest of the subject 102. According to some cases, the mechanical chest compression device 110 is configured to alter one or more chest compression parameters in response to the diastolic pressure being outside of a predetermined range (e.g., greater than the upper threshold or under the lower threshold). Accordingly, the mechanical chest compression device 110 is configured to optimize the administration of the chest compressions based on the diastolic pressure. According to some cases, the mechanical chest compression device 110 is configured to pause the chest compressions in response to the diastolic pressure being outside of the predetermined range. In some implementations, the monitor 106 is configured to transmit an instruction to the mechanical chest compression device 110 in response to determining that the diastolic pressure is outside of the predetermined range. The instruction, for instance, causes the mechanical chest compression device 110 to modify one or more of the chest compression parameters and/or to pause the chest compressions.

In various implementations, the monitor 106 is configured to identify whether the subject 102 has ROSC by analyzing the interpulse interval 120. In some cases, the heart of the subject 102 begins to circulate blood through the body of the subject 102 while the chest compressions are being administered by the mechanical chest compression device 110. However, in some cases, administering additional chest compressions to the subject 102 when the heart is spontaneously circulating blood through the body of the subject 102 can harm the subject 102. For instance, the chest compressions can, in some case, trigger an additional arrhythmia that can trigger an additional instance of cardiac arrest in the subject 102 and prevent the heart of the subject 102 from spontaneously circulating blood. Accordingly, it may be preferred to notify the rescuer 104 and/or the mechanical chest compression device 110 when the heart of the subject 102 is spontaneously circulating blood.

In previous technologies, spontaneous circulation could be identified by detecting and analyzing one or more physiological parameters indicative of circulation during a pause in chest compressions. However, such a pause in chest compressions can cause the subject 102 to develop a hypoxic injury.

According to various implementations of the present disclosure, the monitor 106 is configured to detect ROSC without relying on a pause in chest compressions administered by the mechanical chest compression device 110. When the subject 102 has spontaneous circulation, variability across different instances of the interpulse interval 120 of the blood pressure waveform 114 increases with respect to time. Because the blood pressure waveform 114 becomes reflective of a combination of spontaneous heart beats and chest compressions, the consistency of patterns along a single or multiple instances of the interpulse interval 120 will decrease.

According to some cases, the monitor 106 detects spontaneous circulation by comparing multiple portions of the same interpulse interval 120. For example, the monitor 106 identifies the portion 124 of the interpulse interval 120 and an additional portion of the interpulse interval 120. The additional portion can overlap the portion 124 or may be nonoverlapping with the portion 124. In various cases, the additional portion is in a range of 0.01% to 99.9% of the interpulse interval 120, such as in a range of one third to two-thirds of the interpulse interval 120. The monitor 106 compares the multiple portions of the interpulse interval 120. Based on the comparison, the monitor 106 is configured to detect whether the heart of the subject 102 is spontaneously circulating blood when the mechanical chest compression device 110 is administering the chest compressions. In particular cases, the monitor 106 determines a difference between the mean of the portion 124 and a mean of the other portion of the interpulse interval 120. The monitor 106, in some cases, concludes that the subject 102 has spontaneous circulation in response to determining that the difference between the means is greater than a threshold.

In some examples, the monitor 106 detects spontaneous circulation by comparing portions of different instances of the interpulse interval 120. For instance, the monitor determines whether the subject 102 has spontaneous circulation by comparing the portion 124 of a first instance of the interpulse interval 120 to a portion of a second instance of the interpulse interval 120, wherein the first and second instances of the interpulse interval 120 occur due to separate decompression intervals 116 administered at different times. In various cases, the portion of the second instance of the interpulse interval 120 is in a range of 0.01% to 99.99% of the second instance of the interpulse interval 120, such as in a range of one third to two-thirds of the second instance of the interpulse interval 120. Based on the comparison, the monitor is configured to detect when the heart of the subject 102 is spontaneously circulating blood when the mechanical chest compression device 110 is administering the chest compressions. For example, the monitor 106 determines a difference between the mean of the portion 124 of the first instance of the interpulse interval 120 and a mean of the portion of the second instance of the interpulse interval 120. If the difference is greater than a threshold, the monitor 106 concludes that the subject 102 has spontaneous circulation, for instance.

In various cases, the monitor 106 outputs an indication of the spontaneous circulation to the rescuer 104, such as by visually presenting the indication of spontaneous circulation on the display and/or by audibly outputting the indication of spontaneous circulation via the speaker(s). In some implementations, the monitor 106 outputs the indication to the mechanical chest compression device 110, which is configured to pause the chest compressions in response. In some examples, the monitor 106 outputs, to the mechanical chest compression device 110, an instruction to pause the chest compressions in response to detecting the spontaneous circulation of blood in the body of the subject 102. Accordingly, the chest compressions can be efficiently discontinued when they are no longer needed to prevent the subject 102 from developing a hypoxic injury.

Although FIG. 1 is primarily described with reference to chest compressions administered by the mechanical chest compression device 110, implementations of the present disclosure are not so limited. For example, if manual chest compressions are being administered to the subject 102 by the rescuer 104, the chest compression sensor 112 is configured to detect parameters of the chest compressions being administered to the subject 102. For instance, the chest compression sensor 112 is configured to detect the decompression interval 116 of the manual chest compressions. In various cases, the monitor 106 is configured to detect the diastolic pressure and/or spontaneous circulation based, at least in part, on the parameters detected by the chest compression sensor 112. Moreover, the monitor 106, in some cases, is configured to instruct the rescuer 104 to change one or more chest compression parameters and/or pause the chest compressions based on the diastolic pressure and/or spontaneous circulation of the subject 102.

Moreover, although FIG. 1 is primarily described with reference to determining blood pressure of the subject 102, implementations are not so limited. In some cases, various techniques described herein can also be used to identify a clinically relevant blood flow parameter (e.g., a volumetric blood flow through the blood vessel, a velocity of blood flowing through the blood vessel, or the like) during the interpulse interval 120. In various cases, the blood pressure sensor 108 includes a blood flow sensor (e.g., an ultrasound transducer) configured to sample the instantaneous blood flow parameter through the blood vessel. In various cases, the monitor 106 is configured to determine a mean of the blood flow parameter across at least one portion of the interpulse interval 120. The monitor 106, in some cases, selectively reports the mean of the blood flow parameter. In some examples, the monitor 106 identifies that the subject 102 has spontaneous circulation by determining that a difference between the means of the blood flow parameter during different portions of the interpulse interval 120 is greater than a threshold. Similar techniques can also be used to evaluate other physiological parameters of the subject 102 while the subject 102 is receiving chest compressions. For instance, various techniques described herein with respect to blood pressure can also be applied to a photoplethysmography waveform.

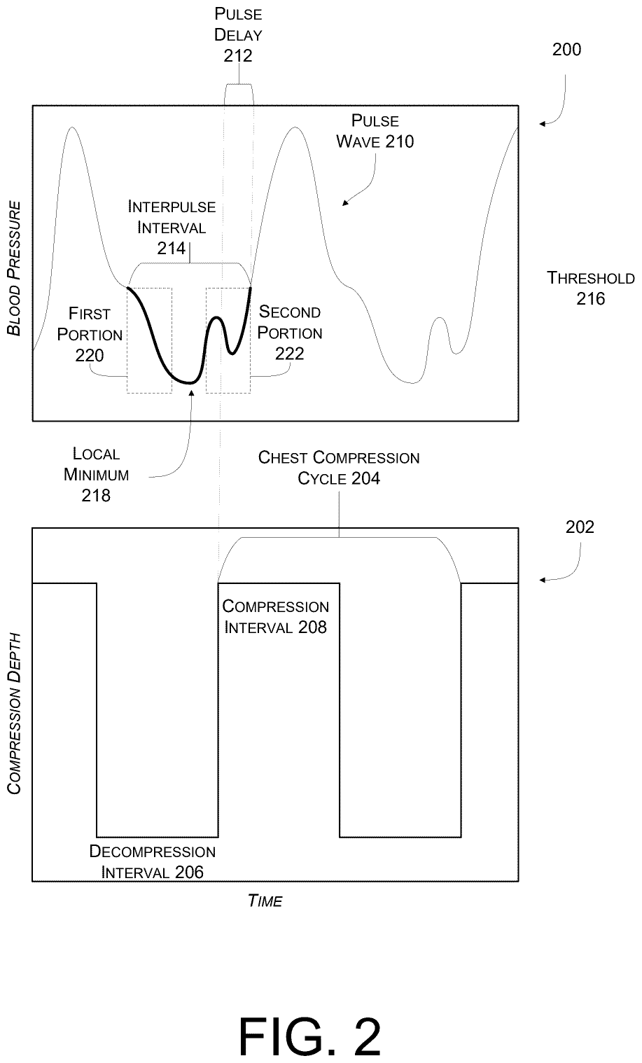

FIG. 2 illustrates a comparison between a blood pressure waveform 200 and a chest compression waveform 202. In FIG. 2, time increases from left to right. The blood pressure waveform 200 represents samples of the instantaneous blood pressure within a blood vessel of a subject while the subject is receiving chest compressions. For example, the blood pressure waveform 200 is defined based on instantaneous blood pressure samples by a blood pressure sensor (e.g., the blood pressure sensor 108) associated with the subject. The chest compression waveform 202 represents the depth of a compressor applying chest compressions to the subject over time. For instance, the subject is the subject 102 receiving the chest compressions from the mechanical chest compression device 110 described above with reference to FIG. 1. In some cases, the chest compression waveform 202 is generated by the device performing the chest compressions (e.g., the mechanical chest compression device 110), a chest compression sensor (e.g., the chest compression sensor 112), or any combination thereof.

The chest compression waveform 202 illustrates multiple chest compression cycles 204. Each chest compression cycle 204 includes a decompression interval 206 and a compression interval 208. During the decompression interval 206, the chest of the subject is released. During the compression interval 208, the chest of the subject is pressed. Because the decompression interval 206 has the same length as the compression interval 208, the chest compression waveform 202 illustrates a 1:1 duty cycle. However, other chest compression duty cycles are also possible.

As the chest of the subject is pressed during the compression interval 208, a pulse wave 210 is generated in the chest of the subject. The pulse wave 210 travels throughout the vasculature of the subject. A velocity of the pulse wave 210 along the blood vessels of the subject is dependent on various factors, including the blood pressure and stiffness of the walls of the blood vessels. In various cases, there is a pulse delay 212 between the compression interval 208 and the detected pulse wave 210. The pulse delay 212, for instance, is dependent on the pulse wave velocity as well as the distance between the chest and the blood vessel from which the instantaneous blood pressure is detected. Due to the pulse delay 212, the compression interval 208 and the pulse wave 210 do not begin and end simultaneously. However, in some cases, a length of the pulse wave 210 is equivalent to a length of the compression interval 208.

An interpulse interval 214 extends between instances in which the pulse wave 210 is detected at the blood vessel. In various cases, the interpulse interval 214 corresponds to the decompression interval 206 of the chest compression cycle 204. However, due to the pulse delay 212, the decompression interval 206 and the interpulse interval 214 do not begin and end simultaneously. In some cases, a length of the decompression interval 206 and a length of the interpulse interval 214 are equivalent.

In various implementations of the present disclosure, the interpulse interval 214 is identified by analyzing the blood pressure waveform 200. In various cases, a threshold 216 defining the transition between the pulse wave 210 and the interpulse interval 214 in the blood pressure waveform 200 is identified. For instance, the threshold 216 is a mean of at least a portion of the blood pressure waveform 200. In some cases, the threshold 216 is a mean of a segment of the blood pressure waveform 200 that has the same length as the chest compression cycle 204. For example, the mean is calculated by determining an integral (e.g., an area under the curve) of the blood pressure waveform 200 from a first time to a second time, wherein an interval between the first time and the second time is equivalent to the length of the chest compression cycle 204. The threshold 216, for instance, is calculated by dividing the integral by the interval between the first time and the second time. According to some cases, the threshold 216 is periodically redefined, such as for every chest compression cycle 204, every five instances of the chest compression cycle 204, every ten instances of the chest compression cycle 204, or the like. In various cases, the interpulse interval 214 is defined as a portion of the blood pressure waveform 200 that begins when the blood pressure waveform 200 dips below the threshold 216 and that ends when the blood pressure waveform 200 rises above the threshold 216.

In some cases, the interpulse interval 214 is derived based on the chest compression waveform 202. For instance, the length of the decompression interval 206 is identified by analyzing the chest compression waveform 202. In examples in which the decompression interval 206 is equivalent in length to the interpulse interval 214, the interpulse interval 214 can be identified based on the length of the decompression interval 206. For example, a center of the interpulse interval 214 may be presumed to be simultaneous with a local minimum 218 of the blood pressure waveform 200. In various cases, the local minimum 218 is the lowest blood pressure between peaks of the blood pressure waveform 200. In various cases, the interpulse interval 214 is identified by centering the length of the decompression interval 206 on the local minimum 218 of the blood pressure waveform 200. For instance, the beginning of the interpulse interval 214 occurs before the local minimum 218 by half of the length of the decompression interval 206, and the ending of the interpulse interval 214 occurs after the local minimum 218 by half of the length of the decompression interval 206. Optionally, the mean of the blood pressure waveform 200 is subtracted from the blood pressure waveform 200 and/or the time corresponding to the center of the interpulse interval 214 is defined as a time of a local minimum of a moving average of the blood pressure waveform 200. In some cases, the moving average of the blood pressure waveform 200 is generated by applying (e.g., convolving) a filter to the blood pressure waveform 200. In some cases, the filter is a boxcar filter. In some examples, the filter is a boxcar filter. In various cases, the filter has a length of the chest compression cycle 204 or a length of the decompression interval 206.

The interpulse interval 214 of the blood pressure waveform 200 is analyzed. In some cases, a diastolic blood pressure is identified by analyzing a first portion 220 or a second portion 222 of the interpulse interval 214. Each of the first portion 220 or the second portion 222 of the interpulse interval 214 includes 0.01% to 0.99% of the interpulse interval 214. In some cases, the first portion 220 or the second portion 222 is one third or two-thirds of the interpulse interval 214. In some examples, the first portion 220 or the second portion 222 includes a single sample of the instantaneous blood pressure captured during the interpulse interval 214. According to various cases, a diastolic blood pressure is determined by calculating a mean of the first portion 220 and/or the second portion 222 of the interpulse interval 214.

In some cases, spontaneous circulation of the subject is identified by comparing the first portion 220 and the second portion 222. For example, if an absolute value of a difference between the mean of the first portion 220 and the mean of the second portion 222 is sufficiently high (e.g., greater than a threshold), spontaneous circulation of the subject can be inferred. In some cases, if an absolute value of a difference between the mean of the first portion 220 or the mean of the second portion 222 and a mean of a portion of another instance of the interpulse interval 214 is sufficiently high (e.g., greater than a threshold), spontaneous circulation of the subject can be inferred. For instance, spontaneous circulation can be identified by analyzing a single instance of the interpulse interval 214 or by comparing multiple instances of the interpulse interval 214 in the blood pressure waveform 200.

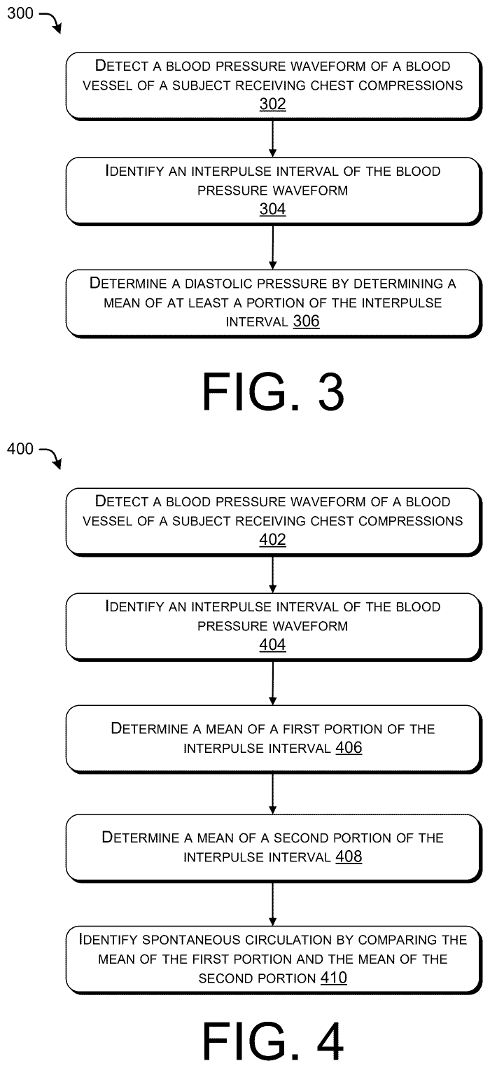

FIG. 3 illustrates an example process 300 for determining a diastolic blood pressure of a subject receiving chest compressions. The process 300 is performed by an entity, in various examples. The entity, for instance, includes a monitor (e.g., the monitor 106), a blood pressure sensor (e.g., the blood pressure sensor 108), a mechanical chest compression device (e.g., the mechanical chest compression device 110), a medical device, a computing device, at least one processor, or any combination thereof.

At 302, the entity detects a blood pressure waveform of a blood vessel of the subject receiving chest compressions. In some cases, the entity generates the blood pressure waveform based on measurements of instantaneous blood pressure from the blood vessel. In some cases, the instantaneous blood pressure is detected by an invasive blood pressure sensor connected to the blood vessel. For instance, the invasive blood pressure sensor includes a catheter inserted into the blood vessel. In some examples, a non-invasive blood pressure sensor is configured to detect the instantaneous blood pressure. For instance, the sensor is configured to output a light or ultrasound signal to blood in the blood vessel, detect a reflection of the light or ultrasound signal from the blood, and determine the instantaneous blood pressure in the blood vessel by comparing the light or ultrasound signal, as transmitted, with its reflection. In some cases, the entity calculates the blood pressure based on the instantaneous blood velocity in the blood vessel, a pulse wave velocity of the blood vessel, and one or more additional parameters.

At 304, the entity identifies an interpulse interval of the blood pressure waveform. The interpulse interval, for instance, occurs between when consecutive pulse waves are detected by the entity at the blood vessel. The pulse waves, for instance, are transmitted along the blood vessel and caused by the chest compressions administered to the subject. Optionally, the entity subtracts a mean of the blood pressure waveform from the blood pressure waveform and/or applies a filter (e.g., a boxcar filter) to the blood pressure waveform. For example, the filter is a boxcar filter having a length of the cycle of the chest compressions (or the decompression interval of the chest compressions) applied to the subject.

According to some cases, the entity identifies a center of the interpulse interval by identifying a local minimum in the blood pressure waveform. In various cases, the entity determines a length of the interpulse interval by determining a length of a decompression interval of the chest compressions. According to some cases, the entity determines the length of the decompression interval by detecting a time between when pressure of the chest compressions is applied to the chest of the subject. In some examples, the entity determines the length of the decompression intervals by receiving a length of the decompression intervals from a chest compression device. In some cases, the entity determines the decompression intervals as a predetermined fraction of a chest compression cycle being applied to the subject. For instance, if the chest compressions are applied at a particular duty cycle (e.g., a 1:1 duty cycle), then the decompression intervals can be identified by dividing the chest compression cycle by a predetermined amount (e.g., 2).

In some implementations, the entity determines a beginning of the interpulse interval by determining a time at which the blood pressure waveform transitions from above a threshold to below a threshold. For example, the entity determines an ending of the interpulse interval by determining a time at which the blood pressure waveform transitions from below the threshold to above the threshold. In some cases, the threshold is a mean of the blood pressure waveform.

At 306, the entity determines a diastolic pressure by determining a mean of at least a portion of the interpulse interval. In some cases, the portion is in a range of about 0.01% to about 99.99%, or a range of 0.1% to 99.9% of the interpulse interval. For example, the portion can be a single instantaneous blood pressure sample detected during the interpulse interval or all but one instantaneous blood pressure sample detected during the interpulse interval. In some cases, the portion is in a range of a third of the interpulse interval to two-thirds of the interpulse interval. According to some cases, the portion begins simultaneously with the interpulse interval (e.g., the portion is an initial portion of the interpulse interval). In some examples, the portion ends simultaneously with the interpulse interval (e.g., the portion is a final portion of the interpulse interval).

In various implementations, the entity outputs an indication of the diastolic pressure. In some cases, the entity generates an alert based on a comparison of the diastolic pressure to one or more thresholds. According to some cases, the entity generates an instruction based on the diastolic pressure. For example, if the diastolic pressure is outside of a threshold range, the entity may generate an instruction to change a chest compression parameter of the chest compressions administered to the subject.

FIG. 4 illustrates an example process 400 for identifying spontaneous circulation in a subject receiving chest compressions. The process 400 is performed by an entity, in various examples. The entity, for instance, includes a monitor (e.g., the monitor 106), a blood pressure sensor (e.g., the blood pressure sensor 108), a mechanical chest compression device (e.g., the mechanical chest compression device 110), a medical device, a computing device, at least one processor, or any combination thereof.

At 402, the entity detects a blood pressure waveform of a blood vessel of a subject receiving chest compressions. In some cases, the entity generates the blood pressure waveform based on measurements of instantaneous blood pressure from the blood vessel. In some cases, the instantaneous blood pressure is detected by an invasive blood pressure sensor connected to the blood vessel. For instance, the invasive blood pressure sensor includes a catheter inserted into the blood vessel. In some examples, a non-invasive blood pressure sensor is configured to detect the instantaneous blood pressure. For instance, the sensor is configured to output a light or ultrasound signal to blood in the blood vessel, detect a reflection of the light or ultrasound signal from the blood, and determine the instantaneous blood pressure in the blood vessel by comparing the light or ultrasound signal, as transmitted, with its reflection. In some cases, the entity calculates the blood pressure based on the instantaneous blood velocity in the blood vessel, a pulse wave velocity of the blood vessel, and one or more additional parameters.

At 404, the entity identifies an interpulse interval of the blood pressure waveform. The interpulse interval, for instance, occurs between when consecutive pulse waves are detected by the entity at the blood vessel. The pulse waves, for instance, are transmitted along the blood vessel and caused by the chest compressions administered to the subject. Optionally, the entity subtracts a mean of the blood pressure waveform from the blood pressure waveform and/or applies a filter (e.g., a boxcar filter) to the blood pressure waveform. For example, the filter is a boxcar filter having a length of the cycle of the chest compressions applied to the subject.

According to some cases, the entity identifies a center of the interpulse interval by identifying a local minimum in the blood pressure waveform. In various cases, the entity determines a length of the interpulse interval by determining a length of a decompression interval of the chest compressions. According to some cases, the entity determines the length of the decompression interval by detecting a time between when pressure of the chest compressions is applied to the chest of the subject. In some examples, the entity determines the length of the decompression intervals by receiving a length of the decompression intervals from a chest compression device. In some cases, the entity determines the decompression intervals as a predetermined fraction of a chest compression cycle being applied to the subject. For instance, if the chest compressions are applied at a particular duty cycle (e.g., a 1:1 duty cycle), then the decompression intervals can be identified by dividing the chest compression cycle by a predetermined amount (e.g., 2).

In some implementations, the entity determines a beginning of the interpulse interval by determining a time at which the blood pressure waveform transitions from above a threshold to below a threshold. For example, the entity determines an ending of the interpulse interval by determining a time at which the blood pressure waveform transitions from below the threshold to above the threshold. In some cases, the threshold is a mean of the blood pressure waveform.

At 406, the entity determines a mean of a first portion of the interpulse interval. In some cases, the first portion is in a range of about 0.01% to about 99.99%, or a range of 0.1% to 99.9% of the interpulse interval. For example, the first portion can be a single instantaneous blood pressure sample detected during the interpulse interval or all but one instantaneous blood pressure sample detected during the interpulse interval. In some cases, the first portion is in a range of a third of the interpulse interval to two-thirds of the interpulse interval. According to some cases, the first portion begins simultaneously with the interpulse interval (e.g., the first portion is an initial portion of the interpulse interval). In some examples, the first portion ends simultaneously with the interpulse interval (e.g., the first portion is a final portion of the interpulse interval).

At 408, the entity determines a mean of a second portion of the interpulse interval. In some cases, the second portion is in a range of about 0.01% to about 99.99%, or a range of 0.1% to 99.9% of the interpulse interval. For example, the second portion can be a single instantaneous blood pressure sample detected during the interpulse interval or all but one instantaneous blood pressure sample detected during the interpulse interval. In some cases, the second portion is in a range of a third of the interpulse interval to two-thirds of the interpulse interval. According to some cases, the second portion begins simultaneously with the interpulse interval (e.g., the second portion is an initial portion of the interpulse interval). In some examples, the second portion ends simultaneously with the interpulse interval (e.g., the second portion is a final portion of the interpulse interval). In some examples, the second portion overlaps with the first portion. In some cases, the second portion is nonoverlapping with the first portion.

At 410, the entity identifies spontaneous circulation by comparing the mean of the first portion and the mean of the second portion. For instance, the entity determines a difference between the mean of the first portion and the mean of the second portion. If the difference (or an absolute value of the difference) is greater than a threshold, the entity may infer that the subject has spontaneous circulation. According to some cases, the entity further compares the mean of the first portion or the mean of the second portion to a mean of a portion of another interpulse interval. If the difference (or an absolute value of the difference) is greater than the threshold, the entity may infer that the subject has spontaneous circulation. In various cases, the entity outputs an indication of the spontaneous circulation. For instance, the entity generates and outputs an instruction to pause the chest compressions in response to detecting that the subject has spontaneous circulation.

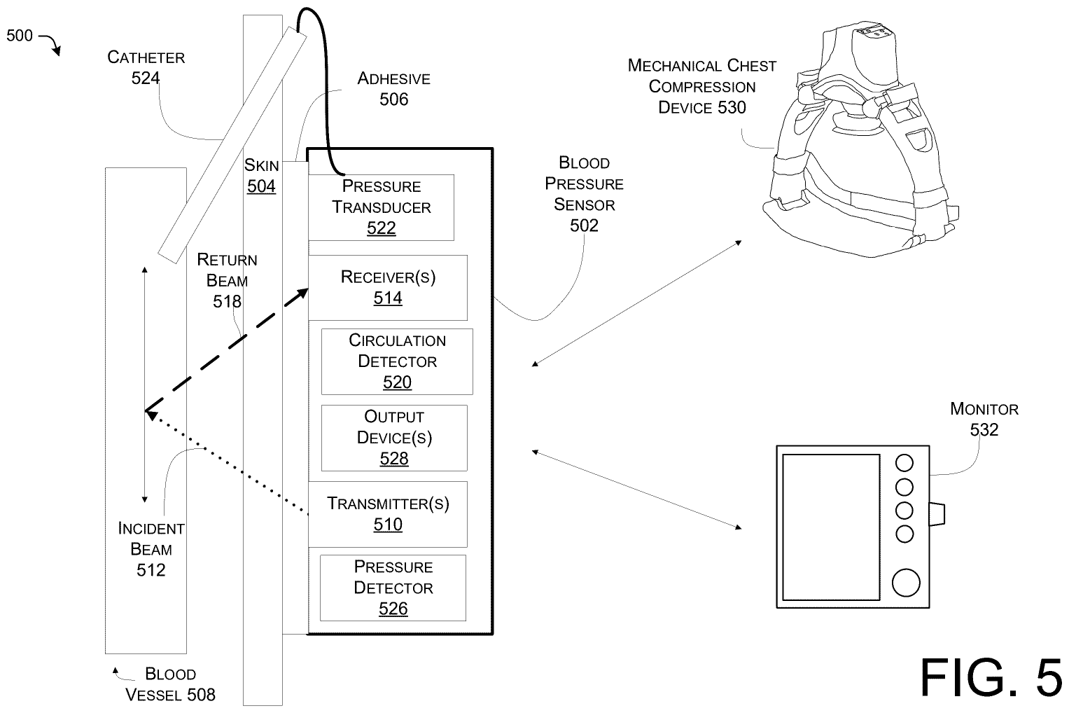

FIG. 5 illustrates an example of a blood pressure sensor 502 configured to detect an instantaneous blood pressure of a subject. For instance, the blood pressure sensor 502 is the blood pressure sensor 108 described above with reference to FIG. 1. FIG. 5 illustrates an example environment 500 for monitoring blood flow through one or more blood vessels of a subject. A blood pressure sensor 502 is adhered to skin 504 of the subject via an adhesive 506. The blood pressure sensor 502, for instance, is located outside of the body of the subject. In some implementations, the blood pressure sensor 502 is held on the skin 504 by a strap, a buckle, a bandage, or some other fastener. For instance, the blood pressure sensor 502 may be wrapped around an extremity of the subject.

In various implementations, a blood vessel 508 is disposed underneath the skin 504. The blood vessel 508 part of the circulatory system of the subject. The circulatory system includes a fluid circuit of various blood vessels (including the blood vessel 508). The subject includes a heart that, when functioning, pumps blood through the blood vessels. In particular, the heart moves blood from lungs of the subject, where the blood can be oxygenated, to other portions of the subject's body, such as the brain, other organs, and the subject's extremities. Along the circulatory system, cells within the blood deliver oxygen to cells of the subject, thereby supporting cellular respiration. In various cases, the blood vessel 508 is an artery that carries oxygenated blood from the lungs of the subject. In some examples, the blood vessel 508 is a vein that carries deoxygenated blood toward the lungs. Examples of the artery include a carotid artery, a subclavian artery, a coronary artery, a brachial artery, an iliac artery, a radial artery, a femoral artery, or a pulmonary artery. Examples of the vein include a jugular vein, an iliac vein, a subclavian vein, a cephalic vein, a brachial vein, a basilic vein, a hepatic vein, a radial vein, an ulnar vein, a digital vein, a brachiocephalic vein, a femoral vein, a saphenous vein, a venous arch, or a tibial vein. In some cases, the pulmonary artery carries deoxygenated blood.

According to various implementations of the present disclosure, the blood pressure sensor 502 is configured to detect the flow of blood through the blood vessel 508. In particular cases, the blood pressure sensor 502 includes one or more transmitters 510 configured to output one or more incident beams toward the blood vessel 508. In the example illustrated in FIG. 5, the incident beams include an incident beam 512.

In various cases, the incident beam 512 includes waves that are transmitted through the skin 504. The waves, for example, can be instantiated as light and/or sound. In various cases, the incident beam 512 includes at least one of infrared, near-infrared, or visible light. For instance, the light may have a frequency in a range of 300 GHz-530 THz and a wavelength in a range of 700 nanometers (nm) to 1 millimeter (mm). For instance, the transmitter(s) 510 include one or more light sources, such as light-emitting diodes (LEDs) or lasers. In some examples, the transmitter(s) 510 may include one or more mirrors configured to split the light output by the light source(s), such as for an interferometric analysis. In some cases, at least one receiver 514 includes one or more light sensors, such as at least one of a photodiode, a phototransistor, a photomultiplier tube, a charge-coupled device, a metal-semiconductor-metal photodetector, or a complementary metal oxide semiconductor photodetector.

According to various implementations, the waves include ultrasound. For instance, the blood pressure sensor 502 is an ultrasound-based blood pressure sensor. As used herein, the term “ultrasound,” and its equivalents, can refer to mechanical waves (e.g., in the form of pressure waves) having a frequency in a range of 20 kilohertz (kHz) to 200 megahertz (MHz). Ultrasound, for example, is sound in a frequency that is greater than an upper detection limit of a human ear. In various instances, transmitter(s) 510 include one or more piezoelectric crystals (including, e.g., lead zirconate titanate (PZT), LiNbO₃ (LN), lead magnesium niobate-lead titanate (PMN-PT), or lead indium niobate-lead magnesium niobate-lead titanate (PIN-PMN-PT)). When an electrical current is induced through the piezoelectric crystal(s), the piezoelectric crystal(s) vibrate at a frequency that produces ultrasound. In some examples, the transmitter(s) 510 include one or more micro-electromechanical system (MEMS) devices. In some instances, the transmitter(s) 510 include one or more capacitive micromachined ultrasonic transducers (CMUTs) and/or piezoelectric micromachined ultrasonic transducers (PMUT). Any electrical to mechanical conversion system or material that operates at the ultrasound frequency range would be suitable.

According to various implementations, the blood pressure sensor 502 includes one or more ultrasound transducers. As used herein, the terms “ultrasound transducer,” “ultrasonic transducer,” “transducer,” and their equivalents, may refer to a device that generates or detects ultrasound. For instance, the ultrasound transducer(s) include the transmitter(s) 510 and/or the receiver(s) 514. In some cases, an ultrasound transducer includes a transducer element (e.g., a piezoelectric crystal, MEMS device, or another type of electrical-to-mechanical conversion device), a first electrode disposed on one side of the transducer element, and a second electrode disposed on another side of the transducer element. In various implementations, the ultrasound transducer is configured to produce ultrasound by inducing a current through or a voltage between the first and second electrodes. In some cases, the ultrasound transducer is configured to detect ultrasound by detecting a current through or voltage between the first and second electrodes that is induced when the ultrasound is received by the transducer element. In some cases, the ultrasound transducer is encased in a housing, which may be watertight. The ultrasound transducer, in some examples, further includes a matching layer that is disposed between the transducer element and a surface of the housing from which an incident beam of ultrasound is emitted. The matching layer includes a material having an acoustic impedance that is between the acoustic impedance of the transducer element and an acoustic lens (if one exists or between the transducer and skin). In some implementations, a gel layer is disposed between the housing of the ultrasound transducer and the skin 504 that further matches the impedance between the ultrasound transducer and the body, thereby preventing incident beam 512 from being reflected by an interface containing air between the skin 504 and the ultrasound transducer. In some cases, the adhesive 506 serves as the gel layer.

In some examples, the incident beam 512 is scattered by blood in the blood vessel 508, thereby generating a return beam 518. The receiver(s) 514 are configured to detect the return beam 518.

As illustrated, the blood vessel 508 is substantially parallel to the skin 504. In various implementations of the present disclosure, the transmitter(s) 510 emit the incident beam 512 in a direction that is non-perpendicular and non-parallel to the surface of the blood pressure sensor 502 that is adhered to the skin 504. That is, the transmitter(s) 510 emits the incident beam 512 in an angled fashion. Thus, a component of the incident beam 512 is parallel to the blood flow through the blood vessel 508.

In various implementations, the return beam 518 may represent a frequency and/or phase shift with respect to the incident beam 512 due to the Doppler effect. These shifts occur due to the Doppler effect and may be referred to as “Doppler shifts.”

According to various implementations, the blood pressure sensor 502 determines a velocity of the blood flow through the blood vessel due to a difference between the frequencies of the incident beam 512 and the return beam 518. The transmitter(s) 510, in some cases, operate in a continuous wave Doppler mode (also referred to as “CW Doppler”), and continuously transmit the first incident beam 512 during a monitoring period. The receiver(s) 514, for instance, continuously detect the return beam 518 during the monitoring period. For instance, the blood pressure sensor 502 detects the blood velocity in the blood vessel 508, in-real time, continuously or semi-continuously based on the frequency shifts between the incident beam 512 and the return beam 518.

In various implementations, the blood pressure sensor 502 operates in a pulsed-wave Doppler mode (also referred to as “PW Doppler”). For instance, the blood pressure sensor 502 causes the transmitter(s) 510 to output the incident beam 512 and causes the receiver(s) 514 to detect the return beam 518 as a return pulse. In various cases, a time delay between the output pulses and the return pulses is indicative of the depth of a structure from which the return pulses are reflected. In various implementations, the blood pressure sensor 502 can determine the blood velocity in the blood vessel 508 based on phase shifts between the pulses of the incident beam 512 and the return beam 518.

In some cases, the blood pressure sensor 502 detects the blood velocities at a sampling rate that corresponds to at least twice the component of the velocity range in the direction of the beam pointing angle of the incident beam 512.

In some examples, the blood pressure sensor 502 performs laser Doppler velocimetry in order to detect the blood velocity. In various cases, the blood pressure sensor 502 includes one or more interferometric sensors. For example, the incident beam 512 includes a light beam (e.g., a coherent light beam) that is split (e.g., by one or more mirrors) prior to transmission through the skin 504. The blood pressure sensor 502 may detect the blood velocity in the blood vessel 508 by comparing the return beam 518 to the split beam generated from the incident beam 512. Techniques for interferometric detection of blood velocity can be found in, for example, R. D. Rader, C. M. Stevens and J. P. Meehan, “An Interferometric Blood Flow Measurement Technique-A Brief Analysis,” in IEEE Transactions on Biomedical Engineering, vol. BME-21, no. 5, pp. 293-297, July 1974; Nagahara, et al., Method. Invest. Ophthalmol. Vis. Sci. 2011;52(1):87-92; and Robinson, et al., Sci Rep 13, 8803 (2023), each of which is incorporated by reference herein in its entirety.

In some cases, the blood pressure sensor 502 images a portion of the subject that includes the blood vessel 508. In some cases, the blood pressure sensor 502 generates an image using the return beam 518 using one or more sonographic techniques. In various implementations, the blood pressure sensor 502 generates the image using multiple return beams including the return beam 518. The blood pressure sensor 502, in some implementations, generates the multiple return beams by sweeping the incident beam 512 across a section of the subject being imaged. For example, the blood pressure sensor 502 may generate a real-time image of the subject that includes cross-sections of the blood vessel 508. In some implementations, the blood pressure sensor 502 automatically segments the cross-sections of the blood vessel 508 in the real-time image. In some cases, the blood pressure sensor 502 performs segmentation of a scrolling Doppler image (e.g., Doppler shift in a y-axis is swept in time along an x-axis) to segregate out the Doppler information of the blood vessel 508. Various types of segmentation techniques can be used, such as detection using histogram of oriented gradients (HOG) features, a scale-invariant feature transform (SIFT), Viola-Jones object detection framework, or You Only Look Once (YOLO). Once the cross-sections depicted in the image are identified using image segmentation, the blood pressure sensor 502 can further classify the cross-sections using a support vector machine (SVM). In some cases, the blood pressure sensor 502 uses one or more trained convolutional neural networks (CNNs) to segment and/or classify the cross-sections of the blood vessel 508 depicted in the image. In various cases, the blood pressure sensor 502 may differentiate the cross-section corresponding to the blood vessel 508 from cross-sections depicting other tissues in the subject.

In some cases, the blood pressure sensor 502 determines the volumetric flow rate through the blood vessel 508 by integrating the velocity of the blood through the blood vessel 508 across a cross-sectional area of the blood vessel 508. In various implementations, the blood pressure sensor 502 is configured to detect a net flow volume that flows through the cross-section of the blood vessel 508 during a time period (e.g., a cardiac cycle, a chest compression cycle, a portion of a chest compression cycle, or any combination thereof) by integrating the volumetric flow rate through the blood vessel 508 over the time period.

According to some examples, the blood pressure sensor 502 includes a circulation detector 520 configured to determine whether the subject has spontaneous circulation based on one or more blood flow parameters derived based on the incident beam 512 and/or the return beam 518. In various cases, the circulation detector 520 identifies an interpulse interval of a waveform representing samples of the blood flow parameter (e.g., volumetric flow rate through the blood vessel 508) over time. By analyzing multiple portions of the interpulse interval, the blood pressure sensor 502 may determine whether the subject lacks spontaneous circulation, or has spontaneous circulation, while the subject is receiving chest compressions.

In some cases, the blood pressure sensor 502 is configured to determine an instantaneous blood pressure of the blood vessel 508 of the subject based, at least in part, on one or more blood flow parameters. For example, the instantaneous blood pressure of the blood vessel 508 can be derived based on the blood velocity and pulse wave velocity of the blood vessel 508 using the Water Hammer equation. Examples of techniques for determining blood pressure based on blood velocity are described in U.S. Pub. No. 2023/0043552, U.S. Pat. No. 11,957,504, U.S. Pub. No. 2022/0369942, U.S. Pub. No. 2019/0053779, U.S. Pub. No. 2023/0043552, which are hereby incorporated by reference herein. According to various cases, the blood pressure sensor 502 is configured to detect the pulse wave velocity of a pulse traveling down the blood vessel 508 by identifying movement at different locations along the blood vessel 508. For instance, if at least a portion of the return beam 518 is reflected or otherwise scattered by a first and a second portion of the wall of the blood vessel 508, the blood pressure sensor 502 may be configured to detect movement in first and second portions of the wall by analyzing the return beam 518 using Doppler-based techniques. The pulse wave velocity, for instance, can be estimated by dividing the distance between the portions of the wall of the blood vessel 508 by the time delay between the movement of the first portion of the wall and the second portion of the wall. Because the blood pressure sensor 502 is configured to detect the blood velocity and pulse wave velocity noninvasively using the incident beam 512 and the return beam 518, in some cases, the blood pressure sensor 502 is configured to detect the instantaneous blood pressure noninvasively.

According to some examples, the blood pressure sensor 502 is configured to detect the blood pressure within the blood vessel 508 invasively. For example, the blood pressure sensor 502 includes a pressure transducer 522 configured to detect a fluid pressure within a catheter 524 that has been inserted under the skin 504 and inside of the blood vessel 508. The pressure within the catheter 524 corresponds to the instantaneous blood pressure in the blood vessel 508. A pressure detector 526 is configured to detect the instantaneous blood pressure within the blood vessel 508 based on the return beam 518 detected by the receiver(s) 514 and/or the pressure detected by the pressure transducer 522. In various cases, the pressure detector 526 detects the instantaneous blood pressure at a sampling rate, such as a sampling rate of 10 Hz, 100 Hz, 1 kHz, 10 kHz, 100 kHz, or the like.

In various implementations, the circulation detector 520 is configured to perform one or more analyses based on the instantaneous blood pressure within the blood vessel 508. For instance, a waveform can be defined based on samples of the instantaneous blood pressure detected by the pressure transducer 522 and/or the receiver(s) 514 over time. In various cases, the circulation detector 520 is configured to identify one or more interpulse intervals of the waveform. Further, the circulation detector 520 identifies portions of the interpulse interval(s) of the waveform representing the instantaneous blood pressure over time. The blood pressure sensor 502, in some cases, is configured to detect whether blood is spontaneously circulating in the subject by comparing the portions of the interpulse interval(s). For example, if an absolute value of a difference between a mean of one portion and a mean of another portion of the interpulse interval(s) is greater than a threshold, the blood pressure sensor 502 may be configured to detect that blood is spontaneously circulating in the blood vessel 508, and the subject as a whole. In some cases, the circulation detector 520 is configured to generate an alert or an instruction based on the presence of spontaneous circulation in the subject. For instance, upon detecting spontaneous circulation, the circulation detector 520 may generate an instruction to pause the administration of chest compressions to the subject.

In various cases, the pressure detector 526 is further configured to detect a diastolic pressure of the subject. For example, the pressure detector 526 detects the diastolic pressure by analyzing a portion of the interpulse interval(s). In some cases, the pressure detector 526 estimates the diastolic pressure by calculating a mean of the portion of the interpulse interval(s). The pressure detector 526, in some cases, is configured to generate an alert or instruction based o the diastolic pressure. For instance, the pressure detector 526 may generate an instruction to change one or more parameters of chest compressions being administered to the subject based on a comparison between the diastolic pressure and one or more thresholds.

The blood pressure sensor 502 is configured to communicate various types of information via one or more output devices 528. The output device(s) 528, in some cases, include a display, a speaker, some other device configured to convey information to a user, or a combination thereof. In various implementations, the output device(s) 528 include one or more transmitters (e.g., transceiver) configured to output data to one or more external devices, such as a mechanical chest compression device 530 (e.g., the mechanical chest compression device 110) or a monitor 532 (e.g., the monitor 106). For instance, the output device(s) 528 are configured to output (e.g., present or transmit) data indicating a blood flow parameter, a blood pressure parameter (e.g., diastolic pressure), an indication of spontaneous circulation, an alert, or an instruction, as described elsewhere herein.

Various implementations of the blood pressure sensor 502 are implemented in hardware and/or software. For example, the circulation detector 520 and/or the pressure detector 526 are implemented as analog circuits and/or software executed by one or more processors in the blood pressure sensor 502.

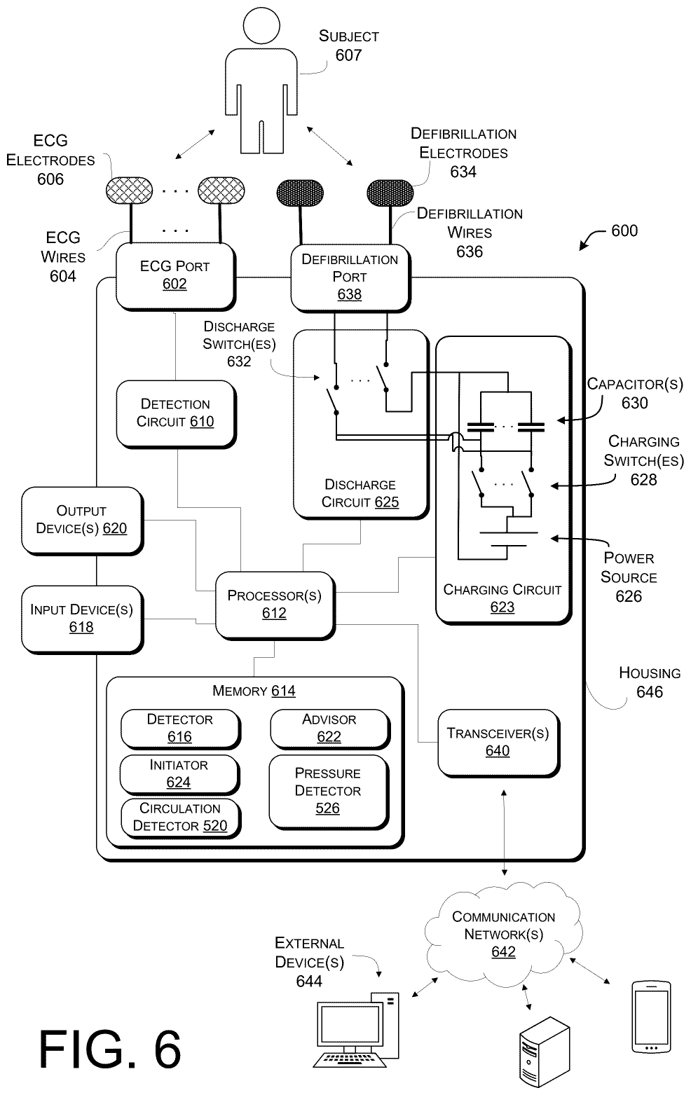

FIG. 6 illustrates an example of an external defibrillator 600 configured to perform various functions described herein. For example, the external defibrillator 600 is the monitor 106 described above with reference to FIG. 1.

The external defibrillator 600 includes an electrocardiogram (ECG) port 602 connected to multiple ECG wires 604. In some cases, the ECG wires 604 are removeable from the ECG port 602. For instance, the ECG wires 604 are plugged into the ECG port 602 via connectors. The ECG wires 604 are connected to ECG electrodes 606, respectively. In various implementations, the ECG electrodes 606 are disposed on different locations on an individual 608. A detection circuit 610 is configured to detect relative voltages between the ECG electrodes 606. These voltages are indicative of the electrical activity of the heart of the individual 608.

In various implementations, the ECG electrodes 606 are in contact with the different locations on the skin of the individual 608. In some examples, a first one of the ECG electrodes 606 is placed on the skin between the heart and right arm of the individual 608, a second one of the ECG electrodes 606 is placed on the skin between the heart and left arm of the individual 608, and a third one of the ECG electrodes 606 is placed on the skin between the heart and a leg (either the left leg or the right leg) of the individual 608. In these examples, the detection circuit 610 is configured to measure the relative voltages between the first, second, and third ECG electrodes 606. Respective pairings of the ECG electrodes 606 are referred to as “leads,” and the voltages between the pairs of ECG electrodes 606 are known as “lead voltages.” In some examples, more than three ECG electrodes 606 are included, such that 5-lead or 12-lead ECG signals are detected by the detection circuit 610.

The detection circuit 610 includes at least one analog circuit, at least one digital circuit, or a combination thereof. The detection circuit 610 receives the analog electrical signals from the ECG electrodes 606, via the ECG port 602 and the ECG wires 604. In some cases, the detection circuit 610 includes one or more analog filters configured to filter noise and/or artifact from the electrical signals. The detection circuit 610 includes an analog-to-digital (ADC) in various examples. The detection circuit 610 generates a digital signal indicative of the analog electrical signals from the ECG electrodes 606. This digital signal can be referred to as an “ECG signal” or an “ECG.”

In some cases, the detection circuit 610 further detects an electrical impedance between at least one pair of the ECG electrodes 606. For example, the detection circuit 610 includes, or otherwise controls, a power source that applies a known voltage (or current) across a pair of the ECG electrodes 606 and detects a resultant current (or voltage) between the pair of the ECG electrodes 606. The impedance is generated based on the applied signal (voltage or current) and the resultant signal (current or voltage). In various cases, the impedance corresponds to respiration of the individual 608, chest compressions performed on the individual 608, and other physiological states of the individual 608. In various examples, the detection circuit 610 includes one or more analog filters configured to filter noise and/or artifact from the resultant signal. The detection circuit 610 generates a digital signal indicative of the impedance using an ADC. This digital signal can be referred to as an “impedance signal” or an “impedance.”

The detection circuit 610 provides the ECG signal and/or the impedance signal one or more processors 612 in the external defibrillator 600. In some implementations, the processor(s) 612 includes a central processing unit (CPU), a graphics processing unit (GPU), both CPU and GPU, or other processing unit or component known in the art.

The processor(s) 612 is operably connected to memory 614. In various implementations, the memory 614 is volatile (such as random access memory (RAM)), non-volatile (such as read only memory (ROM), flash memory, etc.) or some combination of the two. The memory 614 stores instructions that, when executed by the processor(s) 612, causes the processor(s) 612 to perform various operations. In various examples, the memory 614 stores methods, threads, processes, applications, objects, modules, any other sort of executable instruction, or a combination thereof. In some cases, the memory 614 stores files, databases, or a combination thereof. In some examples, the memory 614 includes, but is not limited to, RAM, ROM, electrically erasable programmable read-only memory (EEPROM), flash memory, or any other memory technology. In some examples, the memory 614 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 612 and/or the external defibrillator 600. In some cases, the memory 614 at least temporarily stores the ECG signal and/or the impedance signal.

In various examples, the memory 614 includes a detector 616, which causes the processor(s) 612 to determine, based on the ECG signal and/or the impedance signal, whether the individual 608 is exhibiting a particular heart rhythm. For instance, the processor(s) 612 determines whether the individual 608 is experiencing a shockable rhythm that is treatable by defibrillation. Examples of shockable rhythms include ventricular fibrillation (VF) and ventricular tachycardia (VT). In some examples, the processor(s) 612 determines whether any of a variety of different rhythms (e.g., asystole, sinus rhythm, atrial fibrillation (AF), etc.) are present in the ECG signal.

The processor(s) 612 is operably connected to one or more input devices 618 and one or more output devices 620. Collectively, the input device(s) 618 and the output device(s) 620 function as an interface between a user and the defibrillator 600. The input device(s) 618 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 620 includes at least one of a display, a speaker, a haptic output device, a printer, or any combination thereof. In various examples, the processor(s) 612 causes a display among the input device(s) 618 to visually output a waveform of the ECG signal and/or the impedance signal. In some implementations, the input device(s) 618 includes one or more touch sensors, the output device(s) 620 includes a display screen, and the touch sensor(s) are integrated with the display screen. Thus, in some cases, the external defibrillator 600 includes a touchscreen configured to receive user input signal(s) and visually output physiological parameters, such as the ECG signal and/or the impedance signal.

In various implementations, the input device(s) 618 further include, or are otherwise connected to, one or more physiological sensors. The physiological sensor(s), for instance, are configured to detect one or more physiological parameters of the subject 607. Examples of the physiological sensor(s) include a blood pressure sensor (e.g., a blood pressure cuff, invasive blood pressure sensor, or the like), an airway sensor (e.g., a sensor configured to detect a partial pressure of CO₂ and/or O₂ in an airway of the subject 607), a blood oxygenation sensor (e.g., a pulse oximeter, regional oxygenation sensor, or the like), a thermometer, a pulse sensor, a blood flow sensor (e.g., an ultrasound transducer configured to detect blood flow using Doppler-based techniques), an airway pressure sensor, or any combination thereof. The input device(s) 618, in some cases, includes one or more sensors configured to detect other characteristics of the subject 607. For example, the input device(s) 618 includes an accelerometer, gyroscope, microphone, or any combination thereof. In various implementations, the processor(s) 612 is configured to assess a condition of the subject 607 by analyzing data derived from signals detected by the input device(s) 618. In some examples, the input device(s) 618 include a blood pressure sensor configured to sample an instantaneous blood pressure of a blood vessel of the subject 607.

In some examples, the memory 614 includes an advisor 622, which, when executed by the processor(s) 612, causes the processor(s) 612 to generate advice and/or control the output device(s) 620 to output the advice to a user (e.g., a rescuer). In some examples, the processor(s) 612 provides, or causes the output device(s) 620 to provide, an instruction to perform CPR on the individual 608. In some cases, the processor(s) 612 evaluates, based on the ECG signal, the impedance signal, or other physiological parameters, CPR being performed on the individual 608 and causes the output device(s) 620 to provide feedback about the CPR in the instruction. According to some examples, the processor(s) 612, upon identifying that a shockable rhythm is present in the ECG signal, causes the output device(s) 620 to output an instruction and/or recommendation to administer a defibrillation shock to the individual 608.

The memory 614 also includes an initiator 624 which, when executed by the processor(s) 612, causes the processor(s) 612 to control other elements of the external defibrillator 600 in order to administer a defibrillation shock to the individual 608. In some examples, the processor(s) 612 executing the initiator 624 selectively causes the administration of the defibrillation shock based on determining that the individual 608 is exhibiting the shockable rhythm and/or based on an input from a user (received, e.g., by the input device(s) 618. In some cases, the processor(s) 612 causes the defibrillation shock to be output at a particular time, which is determined by the processor(s) 612 based on the ECG signal and/or the impedance signal.

The processor(s) 612 is operably connected to a charging circuit 623 and a discharge circuit 625. In various implementations, the charging circuit 623 includes a power source 626, one or more charging switches 628, and one or more capacitors 630. The power source 626 includes, for instance, a battery. The processor(s) 612 initiates a defibrillation shock by causing the power source 626 to charge at least one capacitor among the capacitor(s) 630. For example, the processor(s) 612 activates at least one of the charging switch(es) 628 in the charging circuit 623 to complete a first circuit connecting the power source 626 and the capacitor to be charged. Then, the processor(s) 612 causes the discharge circuit 625 to discharge energy stored in the charged capacitor across a pair of defibrillation electrodes 634, which are in contact with the individual 608. For example, the processor(s) 612 deactivates the charging switch(es) 628 completing the first circuit between the capacitor(s) 630 and the power source 626, and activates one or more discharge switches 632 completing a second circuit connecting the charged capacitor 630 and at least a portion of the individual 608 disposed between defibrillation electrodes 634.

The energy is discharged from the defibrillation electrodes 634 in the form of a defibrillation shock. For example, the defibrillation electrodes 634 are connected to the skin of the individual 608 and located at positions on different sides of the heart of the individual 608, such that the defibrillation shock is applied across the heart of the individual 608. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 632 are controlled by the processor(s) 612, for example. In various implementations, the defibrillation electrodes 634 are connected to defibrillation leads 636. The defibrillation wires 636 are connected to a defibrillation port 638, in implementations. According to various examples, the defibrillation wires 636 are removable from the defibrillation port 638. For example, the defibrillation wires 636 are plugged into the defibrillation port 638.

In various implementations, the processor(s) 612 is operably connected to one or more transceivers 640 that transmit and/or receive data over one or more communication networks 642. For example, the transceiver(s) 640 includes a network interface card (NIC), a network adapter, a local area network (LAN) adapter, or a physical, virtual, or logical address to connect to the various external devices and/or systems. In various examples, the transceiver(s) 640 includes any sort of wireless transceivers capable of engaging in wireless communication (e.g., radio frequency (RF) communication). For example, the communication network(s) 642 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 640 includes other wireless modems, such as a modem for engaging in WI-FI®, WIGIG®, WIMAX®, BLUETOOTH®, or infrared communication over the communication network(s) 642.

The defibrillator 600 is configured to transmit and/or receive data (e.g., ECG data, impedance data, data indicative of one or more detected heart rhythms of the individual 608, data indicative of one or more defibrillation shocks administered to the individual 608, etc.) with one or more external devices 644 via the communication network(s) 642. The external devices 644 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 642. In some examples, the external device(s) 644 is located remotely from the defibrillator 600, such as at a remote clinical environment (e.g., a hospital). According to various implementations, the processor(s) 612 causes the transceiver(s) 640 to transmit data to the external device(s) 644. In some cases, the transceiver(s) 640 receives data from the external device(s) 644 and the transceiver(s) 640 provide the received data to the processor(s) 612 for further analysis.

In various implementations, the external defibrillator 600 also includes a housing 646 that at least partially encloses other elements of the external defibrillator 600. For example, the housing 646 encloses the detection circuit 610, the processor(s) 612, the memory 614, the charging circuit 623, the transceiver(s) 640, or any combination thereof. In some cases, the input device(s) 618 and output device(s) 620 extend from an interior space at least partially surrounded by the housing 646 through a wall of the housing 646. In various examples, the housing 646 acts as a barrier to moisture, electrical interference, and/or dust, thereby protecting various components in the external defibrillator 600 from damage.

In some implementations, the external defibrillator 600 is an automated external defibrillator (AED) operated by an untrained user (e.g., a bystander, layperson, etc.) and can be operated in an automatic mode. In automatic mode, the processor(s) 612 automatically identifies a rhythm in the ECG signal, makes a decision whether to administer a defibrillation shock, charges the capacitor(s) 630, discharges the capacitor(s) 630, or any combination thereof. In some cases, the processor(s) 612 controls the output device(s) 620 to output (e.g., display) a simplified user interface to the untrained user. For example, the processor(s) 612 refrains from causing the output device(s) 620 to display a waveform of the ECG signal and/or the impedance signal to the untrained user, in order to simplify operation of the external defibrillator 600.

In some examples, the external defibrillator 600 is a monitor-defibrillator utilized by a trained user (e.g., a clinician, an emergency responder, etc.) and can be operated in a manual mode or the automatic mode. When the external defibrillator 600 operates in manual mode, the processor(s) 612 cause the output device(s) 620 to display a variety of information that may be relevant to the trained user, such as waveforms indicating the ECG data and/or impedance data, notifications about detected heart rhythms, and the like.

The memory 614, in various cases, further includes instructions for executing a pressure detector 526 and/or a circulation detector 520. The pressure detector 526, when executed by the processor(s) 612, causes the processor(s) 612 to determine a blood pressure parameter of the subject 607. For instance, the processor(s) 612 determine a diastolic pressure of the subject 607 by identifying and analyzing one or more interpulse intervals of a blood pressure waveform that includes sampled instantaneous blood pressure measurements of the subject 607. In various cases, the circulation detector 520, when executed by the processor(s) 612, causes the processor(s) 612 to determine whether blood is spontaneously circulating in the body of the subject 607 by comparing portions of one or more interpulse intervals of the blood pressure waveform. Various parameters, alerts, and instructions described herein are transmitted by the transceiver(s) 640 over the communication network(s) 642.

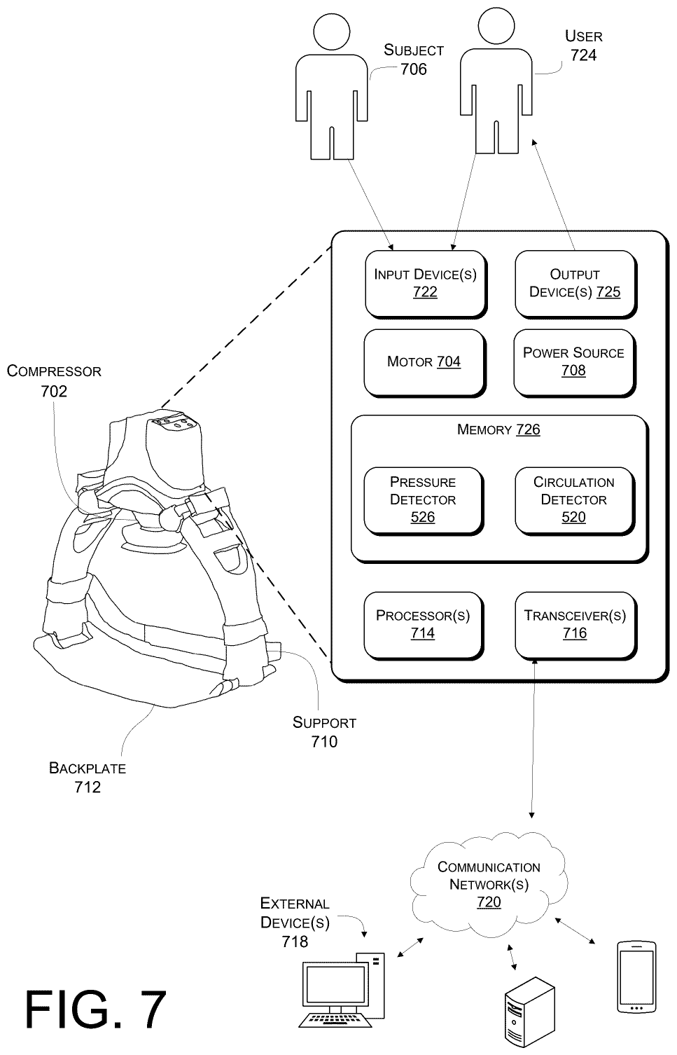

FIG. 7 illustrates a chest compression device 700 configured to perform various functions described herein. For example, the chest compression device 700 is the mechanical chest compression device 110 described with reference to FIG. 1.

In various implementations, the chest compression device 700 includes a compressor 702 that is operatively coupled to a motor 704. The compressor 702 physically administers a force to the chest of a subject 706 that compresses the chest of the subject 706. In some examples, the compressor 702 includes at least one piston that periodically moves between two positions (e.g., a compressed position and a release position) at a compression frequency. For example, when the piston is positioned on the chest of the subject 706, the piston compresses the chest when the piston is moved into the compressed position. A suction cup may be positioned on a tip of the piston, such that the suction cup contacts the chest of the subject 706 during operation. In various cases, the compressor 702 includes a band that periodically tightens to a first tension and loosens to a second tension at a compression frequency. For instance, when the band is disposed around the chest of the subject 706, the band compresses the chest when the band tightens.

The motor 704 is configured to convert electrical energy stored in a power source 708 into mechanical energy that moves and/or tightens the compressor 702, thereby causing the compressor 702 to administer the force to the chest of the subject 706. In various implementations, the power source 708 is portable. For instance, the power source 708 includes at least one rechargeable (e.g., lithium-ion) battery. In some cases, the power source 708 supplies electrical energy to one or more elements of the chest compression device 700 described herein.

In various cases, the chest compression device 700 includes a support 710 that is physically coupled to the compressor 702, such that the compressor 702 maintains a position relative to the subject 706 during operation. In some implementations, the support 710 is physically coupled to a backplate 712, cot, or other external structure with a fixed position relative to the subject 706. According to some cases, the support 710 is physically coupled to a portion of the subject 706, such as wrists of the subject 706.

The operation of the chest compression device 700 may be controlled by at least one processor 714. In various implementations, the motor 704 is communicatively coupled to the processor(s) 714. Specifically, the processor(s) 714 is configured to output a control signal to the motor 704 that causes the motor 704 to actuate the compressor 702. For instance, the motor 704 causes the compressor 702 to administer the compressions to the subject 706 based on the control signal. In some cases, the control signal indicates one or more treatment parameters of the compressions. Examples of treatment parameters include a frequency, timing, depth, force, position, velocity, and acceleration of the compressor 702 administering the compressions. According to various cases, the control signal causes the motor 704 to cease compressions.

In various implementations, the chest compression device 700 includes at least one transceiver 716 configured to communicate with at least one external device 718 over one or more communication networks 720. Any communication network described herein can be included in the communication network(s) 720 illustrated in FIG. 7. The external device(s) 718, for example, includes at least one of a monitor-defibrillator, an AED, an ECMO device, a ventilation device, a patient monitor, a mobile phone, a server, or a computing device. In some implementations, the transceiver(s) 716 is configured to communicate with the external device(s) 718 by transmitting and/or receiving signals wirelessly. For example, the transceiver(s) 716 includes a NIC, a network adapter, a LAN adapter, or a physical, virtual, or logical address to connect to the various external devices and/or systems. In various examples, the transceiver(s) 716 includes any sort of wireless transceivers capable of engaging in wireless communication (e.g., RF communication). For example, the communication network(s) 720 includes one or more wireless networks that include a 3GPP network, such as an LTE RAN (e.g., over one or more LTE bands), an NR RAN (e.g., over one or more NR bands), or a combination thereof. In some cases, the transceiver(s) 716 includes other wireless modems, such as a modem for engaging in WI-FI®, WIGIG®, WIMAX®, BLUETOOTH®, or infrared communication over the communication network(s) 720. The signals, in various cases, encode data in the form of data packets, datagrams, or the like. In some cases, the signals are transmitted as compressions are being administered by the chest compression device 700 (e.g., for real-time feedback by the external device(s) 718), after compressions are administered by the chest compression device 700 (e.g., for post-event review at the external device 718), or a combination thereof.

In various cases, the processor(s) 714 generates the control signal based on data encoded in the signals received from the external device(s) 718. For instance, the signals include an instruction to initiate the compressions, and the processor(s) 714 instructs the motor 704 to begin actuating the compressor 702 in accordance with the signals.

In some cases, the chest compression device 700 includes at least one input device 722. In various examples, the input device(s) 722 is configured to receive an input signal from a user 724, who may be a rescuer treating the subject 706. Examples of the input device(s) 722 include, for instance, at a keypad, a cursor control, a touch-sensitive display, a voice input device (e.g., a microphone), a haptic feedback device (e.g., a gyroscope), or any combination thereof. In various implementations, the processor(s) 714 generate the control signal based on the input signal. For instance, the processor(s) 714 generate the control signal to adjust a frequency of the compressions based on the chest compression device 700 detecting a selection by the user 724 of a user interface element displayed on a touchscreen or detecting the user 724 pressing a button integrated with an external housing of the chest compression device 700.

According to some examples, the input device(s) 722 include one or more sensors. The sensor(s), for example, is configured to detect a physiological parameter of the subject 706. In some implementations, the sensor(s) is configured to detect a state parameter of the chest compression device 700, such as a position of the compressor 702 with respect to the subject 706 or the backplate 712, a force administered by the compressor 702 on the subject 706, a force administered onto the backplate 712 by the body of the subject 706 during a compression, or the like. According to some implementations, the signals transmitted by the transceiver(s) 716 indicate the physiological parameter(s) and/or the state parameter(s).

The chest compression device 700 further includes at least one output device 725, in various implementations. Examples of the output device(s) 725 include, for instance, least one of a display (e.g., a projector, an LED screen, etc.), a speaker, a haptic output device, a printer, or any combination thereof. In some implementations, the output device(s) 725 include a screen configured to display various parameters detected by and/or reported to the chest compression device 700, a charge level of the power source 708, a timer indicating a time since compressions were initiated or paused, and other relevant information.

The chest compression device 700 further includes memory 726. In various implementations, the memory 726 is volatile (such as random access memory (RAM)), non-volatile (such as read only memory (ROM), flash memory, etc.) or some combination of the two. The memory 726 stores instructions that, when executed by the processor(s) 714, causes the processor(s) 714 to perform various operations. In various examples, the memory 726 stores methods, threads, processes, applications, objects, modules, any other sort of executable instruction, or a combination thereof. In some cases, the memory 726 stores files, databases, or a combination thereof. In some examples, the memory 726 includes, but is not limited to, RAM, ROM, EEPROM, flash memory, or any other memory technology. In some examples, the memory 726 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 726 stores instructions, programs, threads, objects, data, or any combination thereof, that cause the processor(s) 714 to perform various functions. In various cases, the memory 726 stores one or more parameters that are detected by the chest compression device 700 and/or reported to the chest compression device 700.

In implementations of the present disclosure, the memory 726 also stores instructions for executing the pressure detector 526 and/or the circulation detector 520. For instance, the chest compression device 700 receives an indication of samples of the instantaneous blood pressure of the subject 706 by a blood pressure sensor among the input device(s) 722 and/or the external device(s) 718. The chest compression device 700, in some cases, is configured to detect the diastolic pressure of the subject 706 and/or to detect whether the subject 706 has spontaneous circulation, simultaneously as the chest compression device 700 is applying chest compressions to the subject 706. In some cases, the chest compression device 700 is further configured to pause the chest compressions, or to change a parameter of the chest compressions, based on the diastolic pressure and/or the presence of circulation.

### EXAMPLE CLAUSES

1. A system, including: a blood pressure sensor configured to detect a blood pressure waveform of an artery of a subject receiving chest compressions, the blood pressure waveform including pulse intervals and an interpulse interval, the pulse intervals corresponding to pulse waves transmitted through the artery and caused by the chest compressions, the interpulse interval extending between the pulse intervals; and a monitor communicatively coupled with the blood pressure sensor and including: a display; and a processor configured to: identify the interpulse interval of the blood pressure waveform; determine a diastolic pressure of the subject by determining a mean of a final two thirds of the interpulse interval of the blood pressure waveform; determine that the chest compressions are inadequate by determining that the diastolic pressure is below a threshold; and cause the display to output an indication of the diastolic pressure and a recommendation to change a parameter of the chest compressions.
2. The system of clause 1, further including: a chest compression sensor communicatively coupled with the monitor and configured to detect a decompression interval of the chest compressions applied to the subject by detecting pressure applied to the chest of the subject, wherein the processor is configured to identify the interpulse interval of the blood pressure waveform by: determining a length of the interpulse interval by analyzing the decompression interval; and determining a beginning of the interpulse interval by detecting a transition of the blood pressure waveform from greater than threshold to lower than the threshold.
3. The system of clause 2, wherein the threshold includes the mean of the blood pressure waveform over a chest compression cycle.
4. The system of any of clauses 1 to 3, wherein the processor is configured to identify the interpulse interval of the blood pressure waveform by: subtracting, from the blood pressure waveform, a mean of the blood pressure waveform; applying, to the blood pressure waveform, a boxcar filter having a length of a cycle of the chest compressions; and identifying a center of the interpulse interval by identifying a local minimum of the blood pressure waveform.
5. A method, including: detecting a blood pressure waveform of a blood vessel of a subject receiving chest compressions; identifying an interpulse interval of the blood pressure waveform, the interpulse interval extending between pulse waves transmitted through the blood vessel and caused by the chest compressions; and determining a diastolic pressure of the subject by determining a mean of the interpulse interval of the blood pressure waveform or a mean of a portion of the interpulse interval of the blood pressure waveform.
6. The method of clause 5, wherein detecting the blood pressure waveform of the blood vessel of the subject receiving the chest compressions includes: detecting the blood pressure waveform by an invasive blood pressure sensor connected to the blood vessel of the subject.
7. The method of clause 5 or 6, wherein detecting the blood pressure waveform of the blood vessel of the subject receiving the chest compressions includes: outputting a light or ultrasound signal to blood in the blood vessel; detecting a reflection of the light or ultrasound signal from the blood in the blood vessel; and determining an instantaneous blood pressure in the blood vessel by comparing the light or ultrasound signal and the reflection of the light or ultrasound signal.
8. The method of any of clauses 6 to 7, wherein identifying the interpulse interval of the blood pressure waveform includes: subtracting, from the blood pressure waveform, a mean of the blood pressure waveform; applying, to the blood pressure waveform, a boxcar filter having a length of a cycle of the chest compressions; and identifying a center of the interpulse interval by identifying a local minimum of the blood pressure waveform.
9. The method of any of clauses 5 to 8, wherein identifying the interpulse interval of the blood pressure waveform includes: identifying a length of the interpulse interval by determining a length of decompression intervals of the chest compressions; and identifying a beginning of the interpulse interval by detecting a transition of the blood pressure waveform from greater than a threshold to lower than the threshold.
10. The method of clause 9, wherein determining the length of decompression intervals of the chest compressions includes: detecting when a pressure of the chest compressions is applied to a chest of the subject; receiving, from a chest compression device administering the chest compressions, an indication of the length of decompression intervals; or determining a chest compression cycle by detecting an interval between local maxima or local minima of the blood pressure waveform, the length of decompression intervals of the chest compressions being a predetermined fraction of the chest compression cycle.
11. The method of any of clauses 5 to 10, wherein the portion of the interpulse interval of the blood pressure waveform is in a range of about a third of the interpulse interval to about two thirds of the interpulse interval.
12. The method of any of clauses 5 to 11, wherein the portion of the interpulse interval of the blood pressure waveform is in a range of about 0.1% of the interpulse interval to about 99.9% of the interpulse interval.
13. The method of any of clauses 5 to 12, wherein the portion of the interpulse interval of the blood pressure waveform includes a single sample of the blood pressure waveform.
14. The method of any of clauses 5 to 13, wherein the portion of the interpulse interval begins with a beginning of the interpulse interval or ends with an ending of the interpulse interval.
15. The method of any of clauses 5 to 14, further including: outputting an indication of the diastolic pressure.
16. A medical device, including: a sensor configured to detect a blood pressure waveform of a blood vessel of a subject receiving chest compressions; and a processor configured to: identify an interpulse interval of the blood pressure waveform, the interpulse interval extending between pulse waves transmitted through the blood vessel and caused by the chest compressions; and determine a diastolic pressure of the subject by determining a mean of a portion of the interpulse interval of the blood pressure waveform.
17. The medical device of clause 16, wherein the sensor includes an invasive blood pressure sensor configured to be connected to the blood vessel of the subject.
18. The medical device of clause 16 or 17, wherein the sensor includes an ultrasound-based blood pressure sensor configured to be disposed on skin of the subject.
19. The medical device of any of clauses 16 to 18, wherein the processor is configured to identify the interpulse interval of the blood pressure waveform by: subtracting, from the blood pressure waveform, a mean of the blood pressure waveform; applying, to the blood pressure waveform, a boxcar filter having a length of a cycle of the chest compressions; and identifying a center of the interpulse interval by identifying a local minimum of the blood pressure waveform.
20. The medical device of any of clauses 16 to 19, wherein the processor is configured to identify the interpulse interval of the blood pressure waveform by: identifying a length of the interpulse interval by determining a decompression interval of the chest compressions; and identifying a beginning of the interpulse interval by detecting a transition of the blood pressure waveform from greater than a threshold to lower than the threshold.
21. The medical device of clause 20, wherein the processor is configured to determine the decompression interval of the chest compressions by: determining a chest compression cycle by detecting an interval between local maxima or local minima of the blood pressure waveform, the decompression intervals of the chest compressions being a predetermined fraction of the chest compression cycle.
22. The medical device of any of clauses 16 to 21, wherein the portion of the interpulse interval of the blood pressure waveform is in a range of about a third of the interpulse interval to about two thirds of the interpulse interval.
23. The medical device of any of clauses 16 to 22, wherein the portion of the interpulse interval of the blood pressure waveform includes: a first third of the interpulse interval; a second third of the interpulse interval; a final third of the interpulse interval; or a first two-thirds of the interpulse interval.
24. The medical device of any of clauses 16 to 23, further including: an output device configured to output an indication of the diastolic pressure.
25. A system, including: a mechanical chest compression device configured to administer chest compressions to a subject; a blood pressure sensor configured to detect a blood pressure waveform of an artery of the subject; and a processor configured to: identify an interpulse interval of the blood pressure waveform, the interpulse interval extending between pulse waves transmitted through the artery and associated with the chest compressions; and determine a mean of a first third of the interpulse interval of the blood pressure waveform; determine a mean of a second third of the interpulse interval of the blood pressure waveform; determine a difference between the mean of the first third of the interpulse interval and the mean of the second third of the interpulse interval; determine that the subject has spontaneous circulation by determining that the difference is greater than a threshold; and in response to determining that the subject has spontaneous circulation, outputting, to the mechanical chest compression device, an instruction to pause the chest compressions.
26. The system of clause 25, wherein the first third of the interpulse interval and the second third of the interpulse interval are temporally overlapping.
27. The system of clause 25 or 26, wherein the first third of the interpulse interval is a final third of the interpulse interval.
28. The system of any of clauses 25 to 27, wherein identifying the interpulse interval of the blood pressure waveform includes: subtracting, from the blood pressure waveform, a mean of the blood pressure waveform; applying, to the blood pressure waveform, a boxcar filter having a length of a cycle of the chest compressions; and identifying a center of the interpulse interval by identifying a local minimum of the blood pressure waveform.
29. The system of any of clauses 25 to 28, the interpulse interval being a first interpulse interval, wherein the processor is further configured to: determining a mean of a portion of a second interpulse interval of the blood pressure waveform, wherein the processor is configured to determine whether the subject has spontaneous circulation further by: comparing the mean of the portion of the second interpulse interval of the blood pressure waveform to the mean of the first third of the first interpulse interval or to the mean of the second third of the second interpulse interval.
30. A method, including: detecting a blood pressure waveform of a blood vessel of a subject receiving chest compressions; identifying an interpulse interval of the blood pressure waveform, the interpulse interval extending between pulse waves transmitted through the blood vessel and caused by the chest compressions; and determining a mean of a first portion of the interpulse interval of the blood pressure waveform; determining a mean of a second portion of the interpulse interval of the blood pressure waveform; and determining whether the subject has spontaneous circulation by comparing the mean of the first portion of the interpulse interval and the mean of the second portion of the interpulse interval.
31. The method of clause 30, wherein detecting the blood pressure waveform of the blood vessel of the subject receiving the chest compressions includes: detecting the blood pressure waveform by an invasive blood pressure sensor connected to the blood vessel of the subject; or detecting the blood pressure waveform by an ultrasound-based blood pressure sensor disposed on skin of the subject.
32. The method of clause 30 or 31, wherein identifying the interpulse interval of the blood pressure waveform includes: subtracting, from the blood pressure waveform, a mean of the blood pressure waveform; applying, to the blood pressure waveform, a boxcar filter having a length of a cycle of the chest compressions; and identifying a center of the interpulse interval by identifying a local minimum of the blood pressure waveform.
33. The method of any of clauses 30 to 32, wherein identifying the interpulse interval of the blood pressure waveform includes: identifying a length of the interpulse interval by determining a length of decompression intervals of the chest compressions; and identifying a beginning of the interpulse interval by detecting a transition of the blood pressure waveform from greater than threshold to lower than the threshold.
34. The method of clause 33, wherein determining the length of decompression intervals of the chest compressions includes: detecting when a pressure of the chest compressions is applied to a chest of the subject; receiving, from a chest compression device administering the chest compressions, an indication of the length of decompression intervals; or determining a chest compression cycle by detecting an interval between local maxima or local minima of the blood pressure waveform, the length of decompression intervals of the chest compressions being a predetermined fraction of the chest compression cycle.
35. The method of any of clauses 30 to 34, wherein the first portion of the interpulse interval of the blood pressure waveform includes: a first third of the interpulse interval of the blood pressure waveform; a second third of the interpulse interval of the blood pressure waveform; a final third of the interpulse interval of the blood pressure waveform; or a final two-thirds of the interpulse interval of the blood pressure waveform, and wherein the second portion of the interpulse interval of the blood pressure waveform is non-overlapping with the first portion of the interpulse interval of the blood pressure waveform.
36. The method of any of clauses 30 to 35, wherein determining whether the subject has spontaneous circulation by comparing the mean of the first portion of the interpulse interval and the mean of the second portion of the interpulse interval includes: determining that a difference between the mean of the first portion of the interpulse interval and the mean of the second portion of the interpulse interval is greater than a threshold; and in response to determining that the difference between the mean of the first portion of the interpulse interval and the mean of the second portion of the interpulse interval is greater than the threshold, determining that the subject has spontaneous circulation.
37. The method of any of clauses 30 to 36, the interpulse interval being a first interpulse interval, the method further including: determining a mean of a portion of a second interpulse interval of the blood pressure waveform, wherein determining whether the subject has spontaneous circulation further includes: comparing the mean of the portion of the second interpulse interval of the blood pressure waveform to the mean of the first portion of the first interpulse interval or to the mean of the second portion of the second interpulse interval.
38. The method of clause 37, wherein determining whether the subject has spontaneous circulation includes determining that the subject has spontaneous circulation, and wherein the method further includes: in response to determining that the subject has spontaneous circulation, outputting an instruction to pause the chest compressions.
39. A medical device, including: a sensor configured to detect a physiological parameter waveform of a subject receiving chest compressions; a processor configured to: identify an interpulse interval of the physiological parameter waveform, the interpulse interval extending between pulse waves transmitted through the subject and caused by the chest compressions; and determine a mean of a first portion of the interpulse interval of the physiological parameter waveform; determine a mean of a second portion of the interpulse interval of the physiological parameter waveform; and determine whether the subject has spontaneous circulation by comparing the mean of the first portion of the interpulse interval and the mean of the second portion of the interpulse interval.
40. The medical device of clause 39, wherein the sensor includes an ultrasound transducer and the physiological sensor includes a blood flow parameter, wherein the sensor includes a blood pressure sensor and the physiological parameter waveform includes a blood pressure waveform, or wherein the sensor includes a blood oxygenation sensor and the physiological parameter waveform includes a photoplethysmography waveform.
41. The medical device of clause 39 or 40, wherein identifying the interpulse interval of the physiological parameter waveform includes: subtracting, from the physiological parameter waveform, a mean of the physiological parameter waveform; applying, to the physiological parameter waveform, a boxcar filter having a length of a cycle of the chest compressions; and identifying a center of the interpulse interval by identifying a local minimum of the physiological parameter waveform.
42. The medical device of any of clauses 39 to 41, wherein identifying the interpulse interval of the physiological parameter waveform includes: identifying a length of the interpulse interval by determining a length of decompression intervals of the chest compressions; and identifying a beginning of the interpulse interval by detecting a transition of the physiological parameter waveform from greater than a threshold to lower than the threshold.
43. The medical device of any of clauses 39 to 42, the sensor being a first sensor, the medical device further including: a second sensor configured to detect when a pressure of the chest compressions is applied to a chest of the subject; or a transceiver configured to receive, from a chest compression device administering the chest compressions, an indication of a decompression interval between the chest compressions.
44. The medical device of any of clauses 39 to 43, wherein the first portion of the interpulse interval of the physiological parameter waveform includes: a first third of the interpulse interval of the physiological parameter waveform; a second third of the interpulse interval of the physiological parameter waveform; a final third of the interpulse interval of the physiological parameter waveform; or a final two-thirds of the interpulse interval of the physiological parameter waveform, and wherein the second portion of the interpulse interval of the physiological parameter waveform is non-overlapping with the first portion of the interpulse interval of the physiological parameter waveform.
45. The medical device of any of clauses 39 to 44, wherein the processor is configured to determine whether the subject has spontaneous circulation by: determining that a difference between the mean of the first portion of the interpulse interval and the mean of the second portion of the interpulse interval is greater than a threshold; and in response to determining that the difference between the mean of the first portion of the interpulse interval and the mean of the second portion of the interpulse interval is greater than the threshold, determining that the subject has spontaneous circulation.
46. The medical device of any of clauses 39 to 45, the interpulse interval being a first interpulse interval, wherein the processor is further configured to: determine a mean of a portion of a second interpulse interval of the physiological parameter waveform, and wherein the processor is configured to determine whether the subject has spontaneous circulation by: comparing the mean of the portion of the second interpulse interval to the mean of the first portion of the first interpulse interval or to the mean of the second portion of the second interpulse interval.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system, comprising:
a blood pressure sensor configured to detect a blood pressure waveform of an artery of a subject receiving chest compressions, the blood pressure waveform comprising pulse intervals and an interpulse interval, the pulse intervals corresponding to pulse waves transmitted through the artery and caused by the chest compressions, the interpulse interval extending between the pulse intervals; and
a monitor communicatively coupled with the blood pressure sensor and comprising:
a display; and
a processor configured to:
identify the interpulse interval of the blood pressure waveform;
determine a diastolic pressure of the subject by determining a mean of a final two thirds of the interpulse interval of the blood pressure waveform;
determine that the chest compressions are inadequate by determining that the diastolic pressure is below a threshold; and
cause the display to output an indication of the diastolic pressure and a recommendation to change a parameter of the chest compressions.

2. The system of claim 1, further comprising:
a chest compression sensor communicatively coupled with the monitor and configured to detect a decompression interval of the chest compressions applied to the subject by detecting pressure applied to the chest of the subject,
wherein the processor is configured to identify the interpulse interval of the blood pressure waveform by:
determining a length of the interpulse interval by analyzing the decompression interval; and
determining a beginning of the interpulse interval by detecting a transition of the blood pressure waveform from greater than threshold to lower than the threshold, and
wherein the threshold comprises the mean of the blood pressure waveform over a chest compression cycle.

3. The system of claim 1, wherein the processor is configured to identify the interpulse interval of the blood pressure waveform by:
subtracting, from the blood pressure waveform, a mean of the blood pressure waveform;
applying, to the blood pressure waveform, a boxcar filter having a length of a cycle of the chest compressions; and
identifying a center of the interpulse interval by identifying a local minimum of the blood pressure waveform.

4. A method, comprising:
detecting a blood pressure waveform of a blood vessel of a subject receiving chest compressions;
identifying an interpulse interval of the blood pressure waveform, the interpulse interval extending between pulse waves transmitted through the blood vessel and caused by the chest compressions; and
determining a diastolic pressure of the subject by determining a mean of the interpulse interval of the blood pressure waveform or a mean of a portion of the interpulse interval of the blood pressure waveform.

5. The method of claim 4, wherein detecting the blood pressure waveform of the blood vessel of the subject receiving the chest compressions comprises:
detecting the blood pressure waveform by an invasive blood pressure sensor connected to the blood vessel of the subject.

6. The method of claim 4, wherein detecting the blood pressure waveform of the blood vessel of the subject receiving the chest compressions comprises:
outputting a light or ultrasound signal to blood in the blood vessel;
detecting a reflection of the light or ultrasound signal from the blood in the blood vessel; and
determining an instantaneous blood pressure in the blood vessel by comparing the light or ultrasound signal and the reflection of the light or ultrasound signal.

7. The method of claim 4, wherein identifying the interpulse interval of the blood pressure waveform comprises:
subtracting, from the blood pressure waveform, a mean of the blood pressure waveform;
applying, to the blood pressure waveform, a boxcar filter having a length of a cycle of the chest compressions; and
identifying a center of the interpulse interval by identifying a local minimum of the blood pressure waveform.

8. The method of claim 4, wherein identifying the interpulse interval of the blood pressure waveform comprises:
identifying a length of the interpulse interval by determining a length of decompression intervals of the chest compressions; and
identifying a beginning of the interpulse interval by detecting a transition of the blood pressure waveform from greater than a threshold to lower than the threshold.

9. The method of claim 8, wherein determining the length of decompression intervals of the chest compressions comprises:
detecting when a pressure of the chest compressions is applied to a chest of the subject;
receiving, from a chest compression device administering the chest compressions, an indication of the length of decompression intervals; or
determining a chest compression cycle by detecting an interval between local maxima or local minima of the blood pressure waveform, the length of decompression intervals of the chest compressions being a predetermined fraction of the chest compression cycle.

10. The method of claim 4, wherein the portion of the interpulse interval of the blood pressure waveform is in a range of about a third of the interpulse interval to about two thirds of the interpulse interval.

11. The method of claim 4, wherein the portion of the interpulse interval of the blood pressure waveform is in a range of about 0.1% of the interpulse interval to about 99.9% of the interpulse interval.

12. The method of claim 4, wherein the portion of the interpulse interval of the blood pressure waveform comprises a single sample of the blood pressure waveform.

13. The method of claim 4, wherein the portion of the interpulse interval begins with a beginning of the interpulse interval or ends with an ending of the interpulse interval.

14. The method of claim 4, further comprising:
outputting an indication of the diastolic pressure.

15. A medical device, comprising:
a sensor configured to detect a blood pressure waveform of a blood vessel of a subject receiving chest compressions; and
a processor configured to:
identify an interpulse interval of the blood pressure waveform, the interpulse interval extending between pulse waves transmitted through the blood vessel and caused by the chest compressions; and
determine a diastolic pressure of the subject by determining a mean of a portion of the interpulse interval of the blood pressure waveform.

16. The medical device of claim 15, wherein the sensor comprises an invasive blood pressure sensor configured to be connected to the blood vessel of the subject, or
wherein the sensor comprises an ultrasound-based blood pressure sensor configured to be disposed on skin of the subject.

17. The medical device of claim 15, wherein the processor is configured to identify the interpulse interval of the blood pressure waveform by:
subtracting, from the blood pressure waveform, a mean of the blood pressure waveform;
applying, to the blood pressure waveform, a boxcar filter having a length of a cycle of the chest compressions; and
identifying a center of the interpulse interval by identifying a local minimum of the blood pressure waveform.

18. The medical device of claim 15, wherein the processor is configured to identify the interpulse interval of the blood pressure waveform by:
identifying a length of the interpulse interval by determining a decompression interval of the chest compressions; and
identifying a beginning of the interpulse interval by detecting a transition of the blood pressure waveform from greater than a threshold to lower than the threshold.

19. The medical device of claim 18, wherein the processor is configured to determine the decompression interval of the chest compressions by:
determining a chest compression cycle by detecting an interval between local maxima or local minima of the blood pressure waveform, the decompression intervals of the chest compressions being a predetermined fraction of the chest compression cycle.

20. The medical device of claim 16, wherein the portion of the interpulse interval of the blood pressure waveform comprises:
a first third of the interpulse interval;
a second third of the interpulse interval;
a final third of the interpulse interval; or
a first two-thirds of the interpulse interval.
